(19) United States
(12) Patent Application Publication
(10) Pub. No.: US 2004/0132017 A1
(43) Pub. Date: Jul. 8, 2004

(54) Oxidoreductase gene associated with the fra16d fragile site

(76) Inventors: Robert Richards, North Adelaide (AU); Karin Reid, North Adelaide (AU); Merran Finnis, North Adelaide (AU); Lynne Hobson, North Adelaide (AU); Marie Mangelsdorf, North Adelaide (AU); Sonia Dayan, North Adelaide (AU); Julie Nancarow, North Adelaide (AU); Erica Woollatt, North Adelaide (AU); Elizabeth Baker, North Adelaide (AU)

Correspondence Address: R Neil Sudol / Henry D Coleman, William J Sapone, Coleman Sudol Sapone, 714 Colorado Avenue, Bridgeport, CT, 06605-1601, US

(21) Appl. No.: 10/149,827

(22) PCT Filed: Dec. 15, 2000
(86) PCT No.: PCT/AU00/01539

(30) Foreign Application Priority Data
Dec. 16, 1999 (AU) PQ 4711
Apr. 19, 2000 (AU) PQ 7025

(51) Int. Cl. C12Q 1/68

(52) U.S. Cl. 435/6

(57) ABSTRACT

The FRA16D fragile site is shown to be located within a gene encoding a protein termed FOR. The fragile site is the location of breakpoints of a variety of chromosomal rearrangements and other mutations associated with tumour cell lines. The FOR protein is shown to be expressed as a number of splice variants. The coding region of the gene encoding FOR protein has been DNA sequenced as has the FRA16D fragile sites. Protein interactive WW domains have been identified as has an oxidoreductase domain. This invention provides for certain diagnostic and potential therapeutic benefits.

### FIELD OF THE INVENTION

This invention relates to the field of cancers and in particular to nucleotide sequences of the fragile site FRA16D, of the FOR gene and amino acid sequences of its encoded proteins, as well as derivatives and analogs thereof and agents capable of binding thereto, and uses of these, such as in diagnosis and therapy.

### BACKGROUND OF THE INVENTION

Cancers are a significant factor in mortality and morbidity, with onset rates of forms of cancer being quite high in all places of the world. Early detection greatly improves the chances of remission and considerably reduces the chance of the cancer metastasizing. The treatment of early stage cancers is also much more benign so that there are less severe residual effects resulting from the treatment. Accordingly early detection of cancers is a high priority in management of the diseases. Similarly treatment of various cancers are of mixed outcome and it is desirable to provide for alternative treatments at least for certain forms of cancers.

Cancers are of many different types and severity, however the uncontrolled proliferation of cancers cells is invariably associated with damaged DNA of one form or another. Some types of cancer are familial in the sense that there is an increased risk of contracting cancer, but the hereditary characteristics in most cancers are not simple and there is only usually a few fold increased risk among family members as compared to the general population. The DNA damage in most cancers are associated with somatic mutations the acquisition of which is thought to be associated with exposure to certain environmental factors.

A very large number of genes have been identified as being associated with the onset of cancer and this reflects the complexity of the regulation of normal cellular proliferation. These genes can be categorised into three groups the first of which includes the so called oncogenes or protooncogenes which are often associated with positive control elements, enhancing cellular proliferation in the normal cellular cycle. Certain mutations in these positive control elements trigger uncontrolled proliferation. A second group are the so called tumour suppressor genes, which are genes that normally suppress proliferation, and inactivation or reduction in activity of these leads to abnormal proliferation. These tend to act in a recessive fashion. A third group are the so-called mutator genes which are normally responsible for maintaining genome integrity during the proliferative cycle, and if these are defective then the general mutation rate increases and the consequent chance of providing for a transforming mutation increases.

One mapping technique to locate the site of chromosomal lesion in a cancer cell is known as the loss of heterozygosity (LOH) technique. Eukaryotes have two copies of each chromosome, apart from the sex chromosomes, and as a result cancers that result from mutations in a tumour suppressor generally require two mutations. Sometimes one mutation will be inherited, and a second mutation is required to trigger the cancer leading to loss of function of both copies of the gene in the individual. Quite often these secondary mutations will be deletions and their location can be detected by checking the presence of highly polymorphic genetic markers from the tumour tissue and from another site such as blood. The markers that are heterozygous in normal tissue and have become homozygous in the cancer tissue can give an indication of the lesion concerned.

The LOH technique is however quite difficult to routinely perform and interpret reliably, this is particularly so because any tumour sample usually is also contaminated by non-tumour tissue, and it is at times difficult to distinguish a result because of a decreased relative intensity, and quantitative amplification techniques will often need to be employed. Another limitation relates to the availability of a suitably dense array of markers which generally leads to the detection only of larger deletions. A single tumour may have LOH in many distinct regions, but LOH will only be detected in those regions that have been tested. The LOH technique is thus unsuited to diagnostic purposes.

The use of these LOH studies have identified a number of sites some of which correspond to regions of the chromosome termed fragile sites.

Fragile sites appear as breaks, gaps or decondensations on metaphase chromosomes. These non-random breaks appear in defined locations on human chomosomes under appropriate conditions.

There are two distinct forms of chromosomal anomaly referred to as fragile sites (Sutherland et al., 1998)). The ‘rare’ form is polymorphic in the population and is accounted for by the expansion of repeat DNA sequences beyond a copy number limit. The ‘common’ form is present at many loci in all individuals. Despite determination of the complete sequence analysis of the common fragile site, FRA3B (Boldog et al., 1996; Inoue et al., 1997; Mimori et al., 1999) and the partial sequence analysis of the common fragile sites, FRA7G and FRA7H (Huang et al., 1998a,b; Mishmar et al., 1998) the molecular basis for common fragile sites is not yet understood.

Fragile sites are also distinguished by the culture conditions required for their induction. Common fragile sites are (mainly) induced by aphidicolin, whereas the rare fragile sites are induced by either high or low concentrations of folate or the AT-rich binding chemicals such as distamycin A or by bromodeoxyuridine. The role of chromosomal fragile sites in human genetic disease was thought to be restricted to fragile X syndrome caused by the FRAXA fragile site, however a mild form of mental retardation has been associated with FRAXE and the FRA11B fragile site appears to predispose to 11q breakage leading to some cases of Jacobsen syndrome.

Fragile sites have been proposed to have a determining role in cancer associated chromosomal instability. There are in excess of 100 fragile sites in the human genome of which the fragile site FRA11B is located within the CBL2 proto-oncogene (Jones et al., 1994, 1995) and the FRA3B, FRA7G and FRA16D sites have been located within or adjacent to regions of instability in cancer cells (Ohta et al., 1996; Sozzi et al., 1996; Engelman et al., 1998; Huang et al., 1998a,b; Chen et al., 1996; Latil et al., 1997).

Recent detailed molecular analysis of fragile site loci has demonstrated that the common fragile site FRA3B is located within a region subject to localised deletion and that this deletion is frequently observed in certain forms of cancer (Ohta et al., 1996; Sozza et al., 1996). FRA3B lies proximal to the major region of LOH on chromosome 3p previously shown to be responsible for deletion of the VHL tumour suppressor (Gnarra et al., 1994). The cancer-associated FRA3B deletions can result in inactivation of a gene (FHIT—Fragile Histidine Triad) which spans the fragile site (Croce et al U.S. Pat. No. 5,928,884). The FHIT gene product has been shown to have a role in tumour growth (Siprashvilli et al., 1997) but quite what the significance or nature of that role is subject of active research at the present.

Another common fragile site FRA 7G has also been shown to be located within an about 1 Mb region of frequent deletion in breast and prostate cancer (18,19) as well as squamous cell carcinomas of the head and neck, renal cell carcinomas, ovarian adenocarcinomas and colon carcinomas (20). The human caveolin-1 and -2 genes are located within the same commonly deleted region as FRA 7G. Caveolin-1 has been shown to have a role in the anchorage dependent inhibition of growth in NIH 3T3 cells (21). The caveolins are therefore candidates for the tumour suppressor gene presumed to be located in the FRA 7G region (20).

Another common fragile site which is aphidicolin inducible is the FRA16D site. FRA16D has been localised at 16q23.2 within a large overlapping region of chromosomal instability in breast and prostate cancer as defined by loss-of-heterozygosity (24,25). One study has found that a significant proportion (77%) of breast cancers carries a deletion at 16q23.2, including the marker D16S518 in the immediate vicinity of FRA16D (24).

There has been no characterisation of a nucleic acid or protein associated with the FRA16D site and the physical location of FRA 16D has not yet been determined. Such a characterisation is desirable to enable potentially early diagnosis and assessment of risk as well as potentially providing for a therapeutic treatment.

### SUMMARY OF THE INVENTION

The inventors have produced a detailed physical map of the FRA16D region which provides markers to identify a relationship between this fragile site and DNA instability in neoplasia and which, further, may allow better diagnosis of cancers associated with the region. This analysis reveals the existence of an intimate relationship between the location of FRA16D and homozygous deletions in various tumours, culminating in the coincidence of two tumour cell DNA breakpoints with the most likely position of the fragile site.

The inventors have also characterised the nucleic acid associated with FRA16D especially by nucleic acid sequencing. Analysis of the DNA sequence and EST sequences associated with the region has identified a number of introns and exons which are found to exist in at least four different splice variants of what will be termed protein FOR. RNA analysis has also been conducted and thus far at least four species of mRNA associated with the region have been detected.

In a first aspect the invention could be said to reside in a method of detecting genetic variations of a 16q23.2 target in the 16q23.2 region of the chromosome, said method comprising the steps of contacting target nucleic acid with one or more oligonucleotides suitable for use as hybridisation probe or PCR priming specific for binding the 16q23.2 specific target, and ascertaining the binding of said oligonucleotide.

It will be understood from the specification that the 16q23.2 specific target might be selected to be within the group comprising the FOR gene, the FRA16D site, or mRNA encoding FOR protein or two or more of these collectively. The target may include chromosomal rearrangements and mutations thereof and the rearrangements or mutations may, in one form, be cancer associated. The variations may include markers in the region such as set forth in this specification including in FIGS. 1, 2 and 6.

The 16q23.2 target within the FOR gene might be selected from one or more of the group comprising exons 1A, 1, 2, 3, 4, 5, 6, 6A, 7, 8, 9, 9A, 10, 10A, 10B or exons located between two adjacent exons or control elements in other adjacent regions that effect an altered expression of the FOR gene. Such adjacent regions may have a promoter, enhancer elements or other regulatory elements. The target may be any one of the splice variants currently identified as FOR I, FOR II, FOR III or FOR IV or it might include other combinations of two or more of the exons.

It is noted in particular that breakpoints of three out of five 16q23.2 translocations associated with multiple myeloma map within the alternate splice of this FOR intron, that is, between exons 8 and 9A, and in one form a preferred target is the intron between exons 8 and 9A or a portion thereof.

In some circumstances the method might be used to detect any rearrangements in a larger target area. Thus it might be desired to use a plurality of oligonucleotides which might be selected to bind to a range of target binding sites within the 16q23.2 specific target to detect for a range of changes. This might be used for example to detect for chromosomal rearrangements such as deletions within the FRA16D site or beyond that in the broader 16q23.2 region. The plurality of oligonucleotides or a plurality of specific binding sites of the 16q23.2 target are preferably spacially separated so that binding of each of the plurality of oligonucleotides or binding to the plurality of specific binding sites can be separately ascertained. The spacial separation might, for example, be conveniently provided as an array on a solid support, for example in a form that is common referred to as a gene chip (see for example patent specifications U.S. Pat. No. 5,288,514 and U.S. Pat. No. 5,593,839). Instead of a plurality of oligonucleotides it may be desired that the target be probed by a single oligonucleotide.

Alternatively the target area might be small, thus for example the method might be used to ascertain the presence or absence of a particular mutation or allelic variation in the 16q23.2 target. Thus for example a target of the 6A, 1A, 9 or 10 or 9A exon will distinguish between FOR I, FOR IV, FOR II and FOR III transcription variants. These may also be used to quantify differences in expression of the splice variants FORII and FORI on the one hand and FORII on the other. It might be expected that because the FORIII only has the WW domains in contrast to FOR II and FOR I a significant biological effect may result from variations in the balance of expression of these different variations of FOR, such variations may give an indication of individuals who are at risk of contracting a form of tumour. A small target area might also be adequate for use with gross chromosomal rearrangements in so far as this might be used to determine the presence or absence of junctions of known chromosomal rearrangements, or alternatively the binding or non binding of one or more of a plurality of oligonucleotides. The target area might also be selected to allow for assessment of the presence or absence of cancer associated point mutations or small DNA rearrangements, using suitably selected oligonucleotides.

The base sequence of the oligonucleotide chosen will depend upon several factors known in the art. Primarily the sequence of the oligonucleotide will be determined by its capacity to bind to the target nucleic acid sequence. The nature of the sequence will depend to some extent on the stringency of the hybridisation required, and whether or not it is desired for one oligonucleotide to detect variation in sequence or not. If variation in one nucleotide is required the stringency of the hybridisation will be high. The length of the oligonucleotide will also be determined by the stringency of the reaction required.

The binding might be by in situ hybridisation of a chromosomal spread, or other suitable spacial arrangement of the target region such as for example on a so called gene chip. Such hybridisation methods will generally provide for an oligonucleotide and be capable of binding the target over a span of at least 15 nucleotides. In the case of hybridisation techniques the oligonucleotides will generally carry a label which can be detected by known measuring methods, especially when bound to the 16q23.2 target. Such labels might include radiolabels such as ³²P or a fluorescent marker.

The method might require a preamplification step whereby the target nucleic acid is amplified, to make it easier to ascertain the binding or non binding of the nucleic acid to the target site.

On the other hand the oligonucleotide might be suitable for amplification of a segment of the target nucleic acid such as by PCR, in which case the size of the target may be somewhat different. With this variation two oligonucleotides might be selected, to provide for amplification of at least part of the target nucleic acid, at least one of the oligonucleotides is required to bind in the target.

The target nucleic acid might be presented in any one of a number of physical forms. Nucleic acid from an individual might be isolated and perhaps digested by a restriction enzyme and spread out such as by electrophoresis on an agarose or polyacrylamide gel, so that binding of the oligonucleotide can be effected whilst the target nucleic acid is supported by the gel or this might be supported on other solid medium such as a gene chip or a metaphase chromosomal spread. Alternatively the oligonucleotide or oligonucleotides might be fixed, and the target nucleic acid might either be diminished in size, or not, and then binding of fragmented targets to the fixed oligonucleotide determined.

The target nucleic acid might be in the form of chromosomal DNA, or might be cDNA or mRNA.

This method might also be used to detect other variants, homologs or analogs of the FRA16D site, FOR gene, or other nucleic acid sequences disclosed in this specification. Thus it might be, for example desirable to determine analagous gene in livestock, domestic, laboratory or sporting animals. Alternatively one might wish to determine another analogous protein that plays a similar role in humans.

In a second aspect the invention relates to a method of detecting the number of alleles for one or more markers in the 16q23.2 target, and this may be a means of perhaps providing a measure of the loss of heterozygosity in an individual. This aspect of the invention therefore relates to locating a deletion that overlaps with the FRA16D region. The method might be achieved by providing a first set of one or more oligonucleotides and a second set of one or more oligonucleotides the first set of oligonucleotide being specific for a first variant of the target nucleic acid, the second set of oligonucleotides being specific for a second variant of the target nucleic acid, the first and second set of oligonucleotides being labelled so as to be capable of being distinguished, and the method comprising the steps of comparing the proportion of binding of the first and second set of oligonucleotides. A method of this sort is set forth in U.S. Pat. No. 5,928,870 to Lapidus et al, which for purposes of practicing the invention is incorporated herein by reference.

It will be understood that the above method is useful in categorising the risk of contracting certain types of cancer associated with the FRA16D fragile site or other portion of the 16q23.2 region.

In a third aspect the invention could be said to reside in a method of determining the level of expression of the FOR gene or any one or more exon thereof, by determining the level of mRNA expression using a probe specific for the FOR gene or exon thereof. This might be used to determine the dysregulation of FOR expression. It will be understood that it may be desired to also determine the level of expression of variants of the gene or exons including rearrangements and mutants including those associated with cancers. This is likely to give a prognosis in relation to at least certain cancers that are currently contracted or perhaps an indication of the risk of contracting one or more types of cancer.

In a fourth aspect the invention could be said to reside in an isolated nucleic acid molecule selected from the group comprising

a) any one or more of the nucleic acids sequences disclosed in the figures hereto or parts thereof

b) FRA16D site

c) FOR gene, or exons thereof

d) mRNA of the FOR gene

e) cDNA of the FOR gene

f) variants of the above including, chromosomal rearrangements and mutations of sequences set out in a) to e) including those variants associated with cancers

g) nucleic acid sequence capable of hybridising specifically to any sequence of a to e above or its complement, and especially those capable of doing so under stringent conditions.

The nucleic acid molecule might include a mosaic from within the above molecules such as a combination of two or more of the group comprising the following, exon 1A, 1, 2, 3, 4, 5, 6, 6A, 7, 8, 9, 9A, 10, 10A, 10B or introns located between two adjacent exons or control elements in other adjacent regions that effect an altered expression of FOR, and it will be understood that such a mosaic includes a molecule encoding cDNA of variants of the FOR protein, whether a wild type allele, a mutated version, or otherwise rearranged. It will thus be understood that the invention includes antisense molecules to any regions of control that might be contemplated above. Such antisense molecules may be used to vary the expression of such protein as are produced by the FOR gene or perhaps adjacent genes such as the c-MAF gene. One may also wish to reduce the expression of one of the splice variants of FOR to provide treatment of a given condition, thus for example it might be desired to have antisense specifically to FOR III if FOR III is overexpressed in the condition.

It will be understood that such nucleic acids include portions of nucleic acids that are suitable for use as primers or probes.

The invention may also be said to include nucleic acids encoding a tumour associated gene from a human or animal capable of hybridizing with any nucleic acid of the fourth aspect of the invention.

In a fifth aspect the invention could be said to reside in a recombinant vector including one or more nucleic acid sequences as set out above, and preferably operably linked to a control element such as might include a functional promoter. The recombinant vector might be used as an expression vector to produce or overproduce FOR protein or variants thereof, or perhaps overproduce nucleic acids associated with the FOR gene such as an antisense molecule. Suitable vectors are generally available commercially or may be constructed as described elsewhere or as is known in the art.

In a sixth aspect the invention could be said to reside in an isolated protein molecule, the protein molecule being selected from the group comprising the following:

a) a FOR protein, or

b) a mutant or variant FOR protein which might optionally be associated with a cancer

In a seventh aspect the invention could be said to reside in a polypeptide produced by any two or more exons selected from the group comprising 1A, 1, 2, 3, 4, 5, 6, 6A, 7, 8, 9, 9A, 10, 10A, 10B joined, said exons being either complete exons or partial, and may be variants.

The invention might also encompass a purified cancer associated protein including a string of amino acids unique to a FOR protein and more particularly as set out in FIG. 9, preferably said amino acid string being at least 10 amino acids long and exhibiting at least 70% amino acid homology more preferably at least 90% homology.

The protein may have an oxidoreductase domain and/or one or more WW domains or may have a role in DNA replication of chromosomal division.

In another form the purified cancer associated protein includes an amino acid string with an amino acid sequence homology of greater than 70% but more preferably greater than 90% with an amino acid string selected from the group comprising:

| | |
|---|---|
| TGANSGIGFETAKSFALHGAHVILACR, | (SEQ ID No 1) |
| LHVLVCNAATFALPWSLTKDGLETTFQVNHLGHFYLVQLLQDVL, | (SEQ ID No 2) |
| YNRSKLCNILFSNELHRRLSPRGVTSNAVHPG | (SEQ ID No 3) |

In another form the purified cancer associated protein includes a WW domain having an amino acid string of 10 amino acid or greater or preferably 20 amino acids or greated with an amino acid sequence homology of greater than 70% but preferably greater than 90% with an amino sequence selected from the group comprising the region 16 to 49 or 57 to 90 of the FOR gene (as graphically illustrated in FIG. 10A), being the amino acid strings

| | |
|---|---|
| DELPPGWEERTTKDGWVYYANHTEEKTQWEHPKT | (SEQ ID No 4) |
| and | |
| GDLPYGWEQETDENGQVFFVDHINKRTTYLDPRL | (SEQ ID No 5) |

In another form the purified cancer associated protein includes at least one oxidoreductase domain having an amino acid string of 10 amino acid or greater or preferably 20 amino acids or greater with an amino acid sequence homology of greater than 70% but preferably greater than 90% with an amino sequence selected from the group comprising the region 130 to 156 or 204 to 247 or 293 to 324 of the FOR gene (as graphically illustrated in FIG. 10A).

In an eighth aspect the invention includes an agent capable of selectively binding a FOR protein or fragment or variant thereof. Such agents may be particularly useful in diagnostic methods. Such an agent may also be used to bind a protein containing a string of amino acids unique to FOR or variant thereof and in particular such variants that are currently known to be associated with one or more forms of cancer. The agent may selectively bind to the variant FOR as compared to an FOR protein not associated with cancer. Such an agent might be an agonist or an antagonist of FOR function. It might therefore be desired to provide for a number of agents each capable of selectively binding to a separate one of a number of variants of FOR so that it is possible to distinguish between variants. Thus for example it might be desired to target the C terminus of respectively FOR I, FOR II, FOR III and FOR IV to distinguish between these four proposed forms. The invention therefore also encompasses a method of detecting variants of the FOR protein. Measuring the relative levels of these four and other forms of FOR protein is likely to give an indication of regulatory perturbations which may be associated with certain cancers.

The nature of the agents can vary depending on their intended use. Thus for a diagnostic method an antibody or fragment thereof, such as an Fab fragment, of a recombined molecule carrying the variable region of an antibody recognising the desired portion of the FOR may be adequate. The antibody might be polyclonal however preferably the antibody is a monoclonal antibody prepared by known techniques.

Alternatively small molecules capable of binding the desired portion of the FOR protein may be used, such small molecules might include peptides, proteins, nucleic acids or sugars or other organic molecules. These can be isolated by screening using known techniques from libraries of suitable compounds. Such small molecules can then be tested for antagonist or agonist properties to potentially provide a therapeutical agent which have the potential to be used in the treatment of cancers. These agents would be administered by clinicians in an appropriate manner.

Also useful therapeutically might be the provision of an isolated protein of the seventh aspect of the invention, particularly those forms that mimic the action of a wild type FOR, and perhaps simply the purified FOR. It is anticipated that the FOR protein in at least one of its forms is a tumour suppressor, that is, its absence increases the risk of aberrant cell division leading to a cancer. Accordingly one form of therapy may include the administration of such a protein to an individual who is considered at risk, particularly if they are found to have an altered FOR protein. Such administration would be in conformity with normal practices in a suitable excipient. It may also be the case that the aberrant FOR protein actively enhances tumourigenesis and accordingly it might be appropriate to administer an antagonist of the aberrant variant at the same time. Alternatively the administration of the antagonist on its own may be of therapeutic benefit. Thus for example FORIII is anticipated to be a competitor of FORII and/or FORI, and thus expression of FORIII at higher or lower levels relative to FORII and/or FORI is likely to have a therapeutic effect.

Another form of treatment which is becoming increasingly contemplated is to provide for a method of gene therapy and one method of undertaking cell therapy is to provide for certain progenitor cells which include incorporated therein a vector capable of producing an appropriate form of FOR protein. Accordingly in a ninth aspect the invention could be said to reside in a recombinant host cell having stably inserted therein DNA of any one of the forms of DNA contemplated in the third aspect of the invention. In preference the DNA is capable of producing a tumour suppressing form of FOR, and most conveniently this will be a wild-type form of FOR, which may simply be a cDNA molecule or the FOR gene. Alternatively however it may also be desired to have a host cell which has a DNA sequence capable of producing an antisense molecule in the case where a tumour promoting form of the FOR molecule is produced by the individual to be treated, the antisense capable of reducing the level of expression of the FOR molecule.

Methods of gene therapy are not limited to cases where the appropriate nucleic acid is delivered in a host cell, but also includes the administration of the nucleic acid specifically to the site of interest.

The recombinant host cell may not necessarily be used for therapeutic purposes, it may also be used for over-expression of the protein, or a nucleic acid associated with FOR, or the 16q23.2 region, and may therefore be bacterial, yeast, plant, animal, preferably mammalian or human. Additionally the invention contemplates the provision of a transgenic non-human animal carrying recombinantly altered or overexpressing 16q23.2 DNA, preferably FRA16D or FOR gene, or other DNA of the fourth form of this invention. The recombinant DNA might be incorporated into the chromosome of the host, alternatively the host cell may carry said recombinant DNA in a self replicating element such as a plasmid.

The agents of the eighth aspect may be used for ascertaining the level of expression of FOR, variants or exons thereof, to determine whether there is an altered level of expression. Thus a western blot using a labelled agent may be used for the purpose using known techniques.

This is another means of measuring dysregulation of expression.

### BRIEF DESCRIPTION OF THE DRAWINGS

FIG. 1: Positional cloning of FRA16D and location of loss of heterozygosity and translocation in cancer.

A. The locations of loss-of-heterozygosity regions in breast and prostate cancer and the approximate location of the FRA16D fragile site are indicated with respect to genetic markers (downward arrows) in the 16q23.2 region. Markers in the vicinity of FRA16D are shaded. The approximate location as determined by Chesi et al. (1) of multiple myeloma breakpoints and the c-MAF gene (bar) are also shown by upward black arrows. Not to scale.

B. Map of the contig of YAC subclones across the FRA16D region with respect to genetic markers and FRA16D. Open boxes indicate those YACs which map by fluorescence in situ hybridisation proximal to FRA16D, grey boxes are those which span FRA16D and black boxes indicate those YACs which map distal to FRA16D. Not to scale.

FIG. 2: Positional cloning of FRA16D and the extent of heterozygous and homozygous deletion in the AGS tumour cell line.

A. Pulsed-Field gel map of ˜1 Mb of the ‘Right Hand Side’ (RHS) of YAC My801B6 and the location of BACs, genetic and STS markers (key markers are boxed). Restriction sites between Afma336yg9 and WI2755 are shown in B. The AGS stomach cancer cell line homozygous deletion is indicated—shaded circles denote the presence and open circles the absence of PCR products for the STS markers. Maximal region of heterozygous deletion in AGS cell line is indicated by polymorphic D16S518 and D16S3029 PCR products, indicated as A and B alleles. The two AGS cell line chromosome 16s are indicated by shaded bars.

B. Restriction map of the critical FRA16D region (Afma336yg9 to D1653029) showing the location of key members of the lambda subclone tile path used for FISH in FIG. 3. Clones designated l-n are from 325M3; others are from 801B6. Open boxes represent those subclones found to map proximal (on the basis that >85% of their FISH signals were proximal to FRA16D), grey boxes those which appear to span the fragile site (less than 85% on one side or other of FRA16D) and black boxes those which are distal to the fragile site (on the basis that >85% of their FISH signals were distal to FRA16D). l clones which gave high background on FISH were not scored. These and other l clones for which FISH data were not obtained are included as thin boxes. STS localisation of the AGS homozygous breakpoints are indicated by the presence (shaded circles) and absence (open circles) of PCR products.

FIG. 3: Fluorescence in situ hybridisation (FISH) of lambda subclones against FRA16D expressing chromosomes.

Each panel contains two FRA16D expressing partial metaphases, with and without FISH signal merged. In each case the width of the gap or break at the fragile site is greater than the width of the chromatid. (a) 1504 showing signal proximal to FRA16D; (b) 1181 showing signal proximal and distal to FRA16D; (c) 1191 (upper) and 18 (lower) showing signal distal to FRA16D. Images of metaphase preparations were captured by a cooled CCD camera using the ChromoScan image collection and enhancement system (Applied Imaging International Ltd.). FISH signals and the DAPI banding pattern were merged for figure preparation.

FIG. 4: Fluorescence in situ hybridisation mapping of the lambda subclone tile path across FRA16D.

The individual lambda clones were scored against chromosomes where the FRA16D gap or break was greater than the chromatid width. Each increment represents a single FISH signal. n=number of chromosomes scored. Scores were plotted as proximal (p) and distal (d) with respect to FRA16D. Maximum location for FRA 16Ds indicated by arrows. Location of BAC clones 325M3 and 353B15 is also shown. The boxed lambda contig subclones indicate those for which FISH signal results with respect to the FRA16D fragile site were obtained—open boxes, had >85% signal proximal to FRA16D; grey boxes, spanning (<85% signal on one side or other of FRA16D) and black boxes, had >85% signal distal to FRA16D. While this figure is not to scale the location of the lambda clones can be determined from their position in FIG. 2. Thin boxed lambda clones are those for which FISH data was not obtained.

FIG. 5: Duplex PCR deletion detection at the FRA16D locus in tumour cell lines.

PCR products from the duplex of STSG-10102 and dystrophin DMD Pm were subjected to agarose gel electrophoresis and ethidium bromide staining. Template DNAs were seven tumour cell lines and blood bank and no DNA controls. Markers are HpalI digested pUC19. The position of the STSG-10102 and DMD Pm PCR products are indicated by large grey-filled arrows while the primer dimer PCR artefact is indicated by a small white arrow.

FIG. 6: A. Extent of loss of heterozygosity regions in breast (25) and prostate cancer (24) in relation to the cytogenetic position of the FRA16D fragile site as determined by fluorescence in situ hybridisation of a tile path of subclones as show in FIG. 4.

B. Map of YACs which span FRA16D region showing approximate location of multiple myeloma breakpoints (MM.1, ANBL6, JJN3) determined by Chesi et al., (1). Location of homozygously deleted regions in AGS and HCT 116 tumour cell lines as determined by STS content. The locations of various partial BAC sequences (as evident by STS content) are indicated. Striped boxes=determined sequence accession numbers.

C. The location of the FRA16D spanning DNA sequence and the respective exons of the alternative spliced FOR gene transcripts (numbered black boxes). Clusters of ESTs sequences representative of each of the alternative mRNA 3′ ends are given.

FIG. 7: A. Northern blots of RNA from various human tissues. Expected FOR mRNAs (I-IV) are indicated for the respective DNA probes which span various exons of the FOR gene. H, heart, Br, brain; Pl, placenta; Lu, lung; Li, liver; sM, skeletal muscle; K, kidney; P, pancreas. Arrows indicate FOR mRNAs (FOR I approx. 1.3 kb, FOR II approx 2.2 kb, FOR III approx 0.74 kb)

B. Northern blots of RNA from various human tissues, spleen, thymus, prostate, testis, ovary, small intestine, colon, peripheral blood leukocytes.

Probes (I, II and III) and (I and II) are as indicated in FIG. 6. FOR I, FOR II and FOR III mRNAs are indicated. Additional transcripts hybridizing to the FOR probes are indicated by grey arrows.

FIG. 8 A. Is a composite DNA sequence of the predicted FOR I transcript (SEQ ID No 28) constructed by conjoining overlapping EST, RT-PCR and 5′ RACE DNA sequences.

B. Is a composite DNA sequence of the predicted FOR II transcript (SEQ ID No 29) constructed by conjoining overlapping EST, RT-PCR and 5′ RACE DNA sequences.

C. Is a composite DNA sequence of the predicted FOR III transcript (SEQ ID No 30) constructed by conjoining overlapping EST, RT-PCR and 5′ RACE DNA sequences.

D. Is a composite DNA sequence of the predicted FOR IV transcript (SEQ ID No 31) constructed by conjoining overlapping EST and RT-PCR DNA sequences.

FIG. 9 are composite amino acid sequences predicted for the sequences for FOR I (SEQ ID No 32), FOR II (SEQ ID No 33), FOR III (SEQ ID No 34) and FOR IV (SEQ ID No 35) as shown in FIG. 8, unique sequences are underlined.

FIG. 10 A. Is a diagrammatic representation of the four FOR amino acid sequences showing the locations of the alternate splice sites, the position of the exons, the three predicted oxido reductase domains, and the predicted WW domains. The sequence numbers refer to the amino acid sequence.

B. Is an alignment of the sequences WW domains (SEQ ID No 4 and SEQ ID No 5) with each other and with the WW domain consensus sequence.

FIG. 11 sets out DNA sequences for each of the exons identified for the FOR protein as well as a small amount of flanking intron sequence. The exon sequences are in uppercase, while the intron sequence is in lower case. Some nucleotide sequences are in bold, splice donor (GT) and acceptor (AG) sites, polyadenylation signals (AATAA) and initiation Methionine (ATG). For exons 1 and 1A an upstream in phase termination codon is in italics and confirms the correct open reading frame in these mRNAs.

FIG. 12 is about 270 kb of DNA sequence that overlaps and defines within it the FRA16D fragile site (SEQ ID No 53), which is shown to reside between exons 8 and 9, this sequence has been deposited in the GenBank database and has been assigned accession number AF217490 as indicated in FIG. 6.

FIG. 13 is DNA sequence deposited with GenBank database and identifed by accession number AF217492 as indicated in FIG. 6, and which encompasses exon 7 (SEQ ID No 52).

FIG. 14 is DNA sequence deposited with GenBank database and identifed by accession number AF217491 as indicated in FIG. 6, and which encompasses exon 6 (SEQ ID No 51).

FIG. 15 shows FOR transcripts in normal and tumour cells. Products that were subjected to sequence analysis are indicated by arrowheads.

A RT-PCR were either ‘specific’ for the FOR III transcript or ‘general’ being able to detect FOR I-III mRNAs.

B 5′RACE specific for the FOR I, FOR II and FOR III transcripts in ‘normal’ HS578BST cells and T47D tumour cells.

### DETAILED DESCRIPTION OF THE INVENTION

### EXAMPLE 1

### Mapping of the FRA 16D Fragile Site

Materials and Methods

Isolation of DNA Probes and YACs in the FRA16D Region

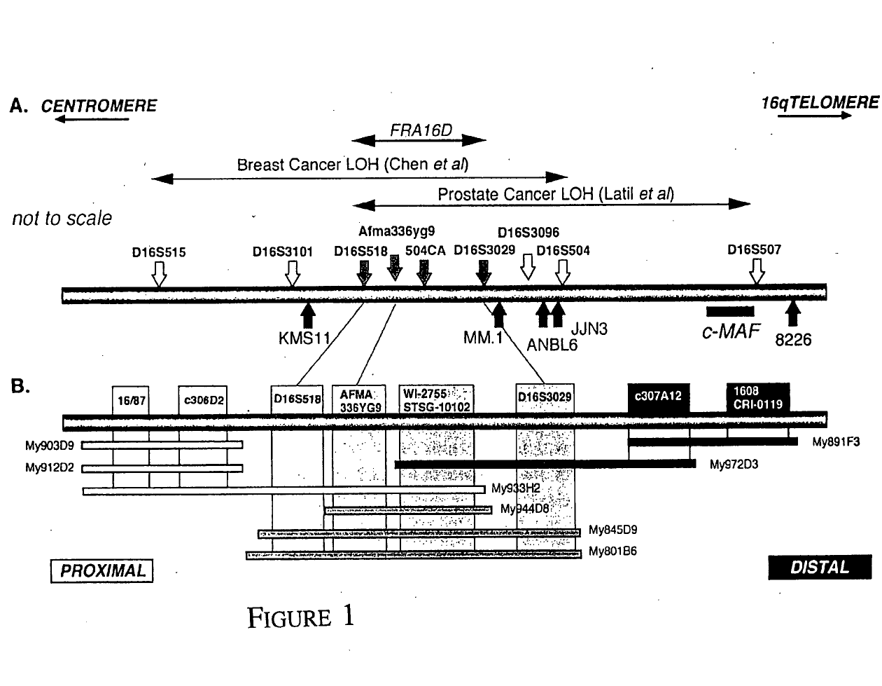

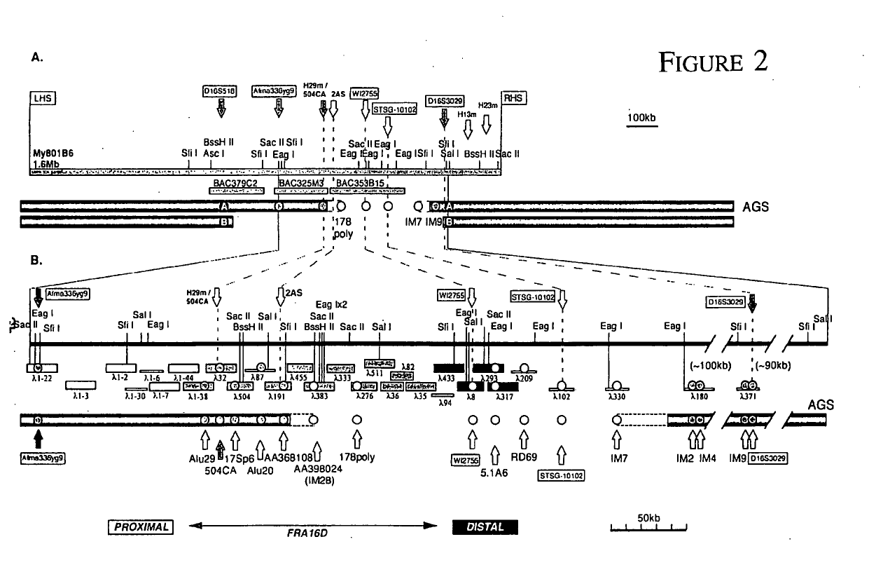

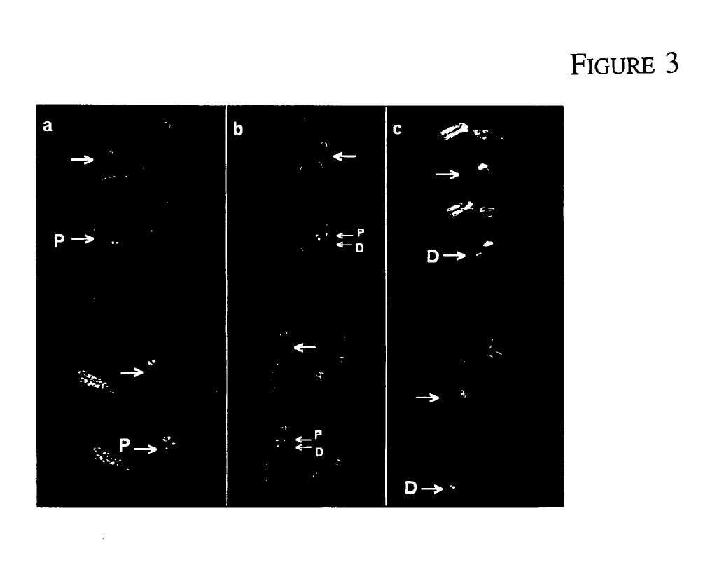

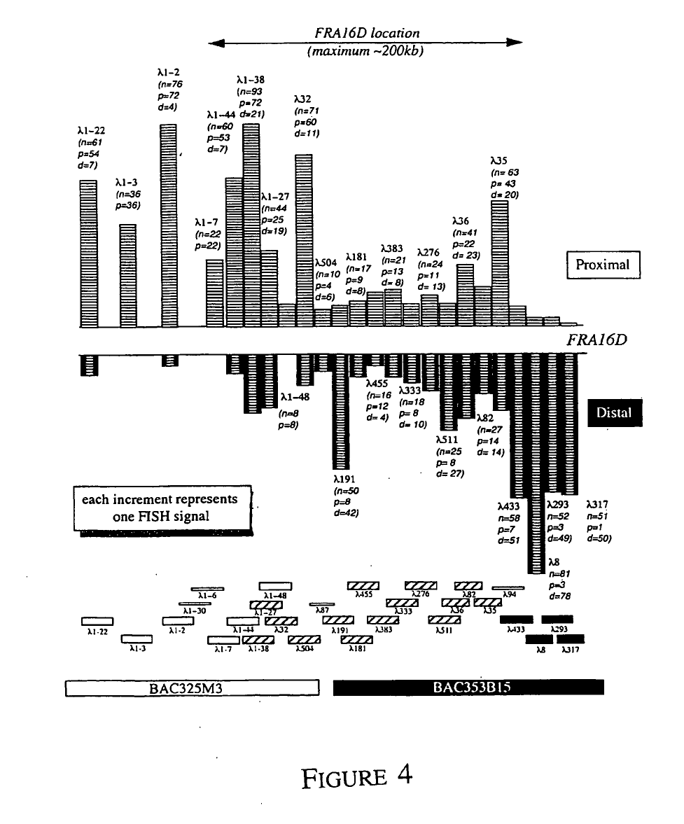

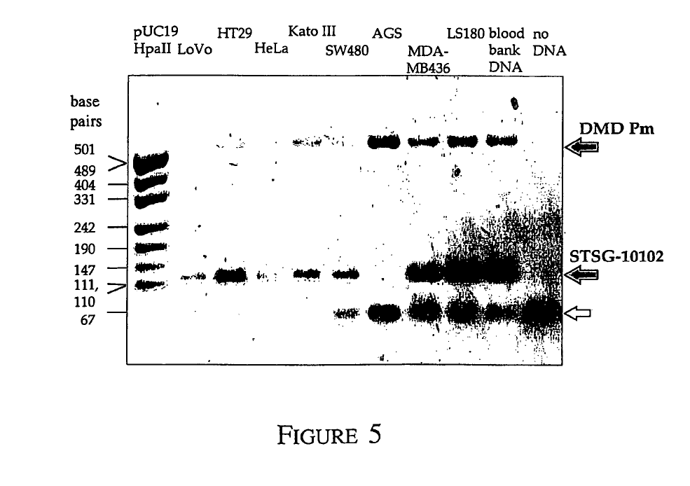

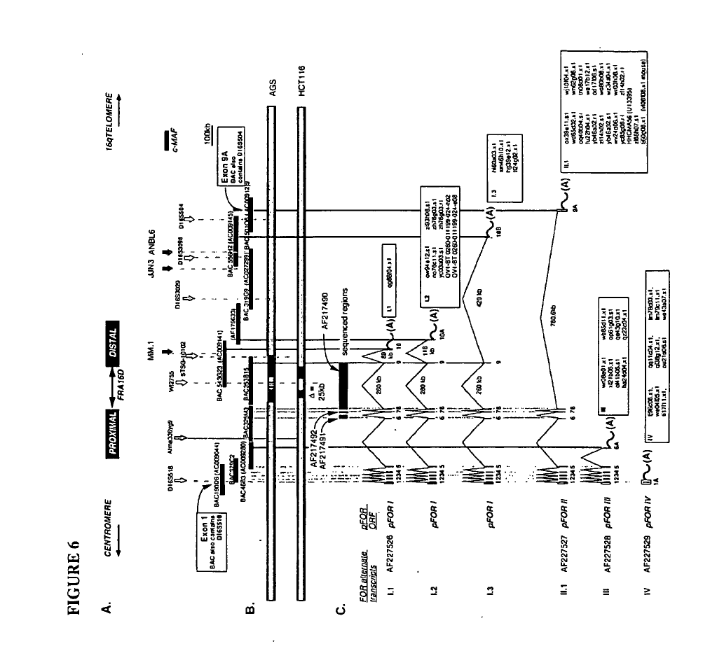

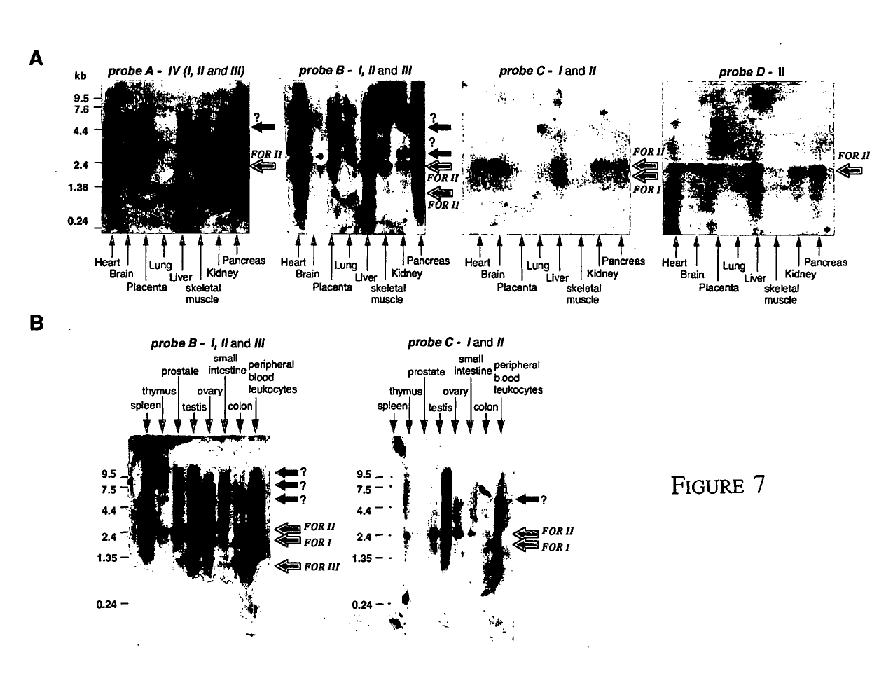

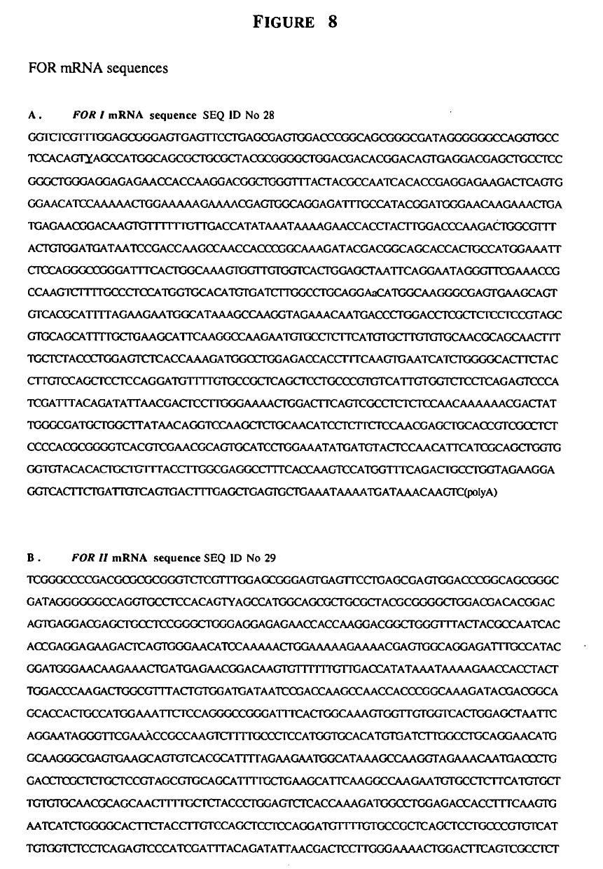

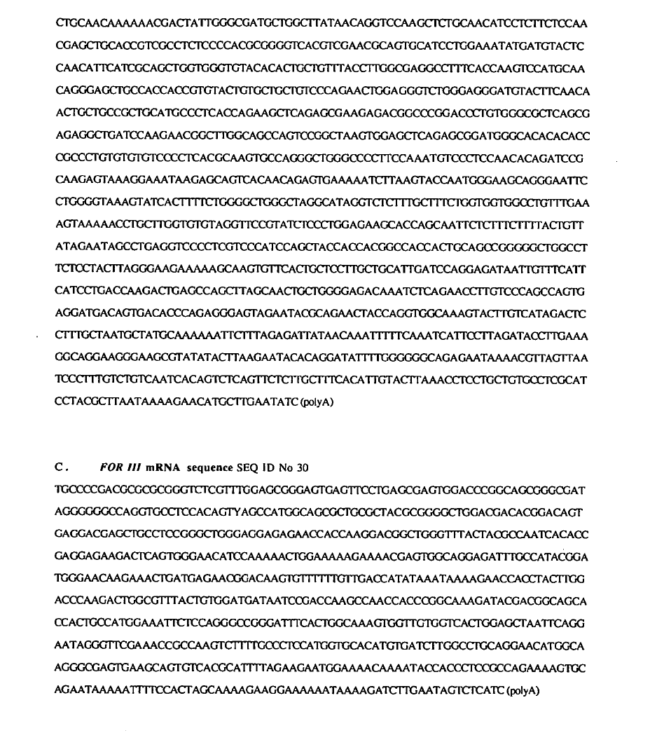

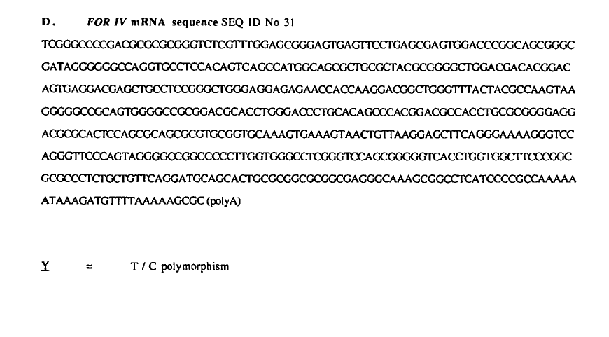

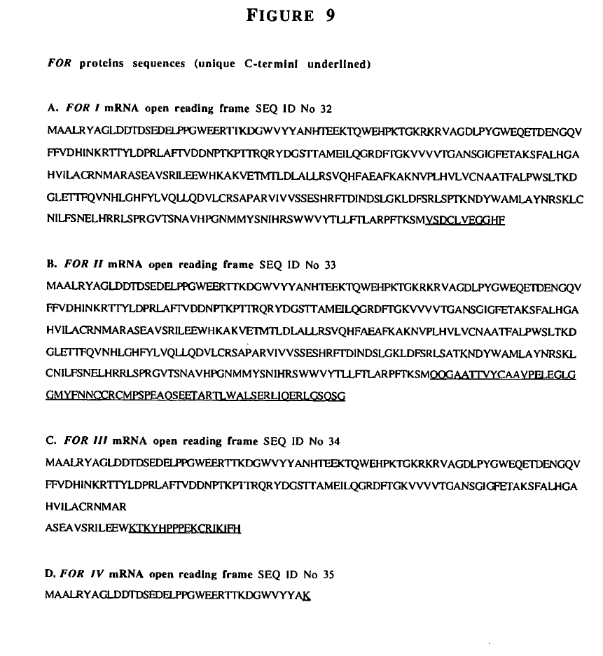

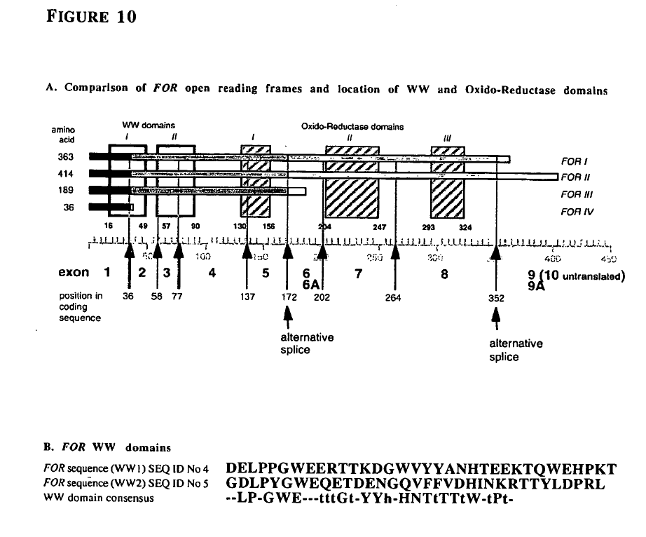

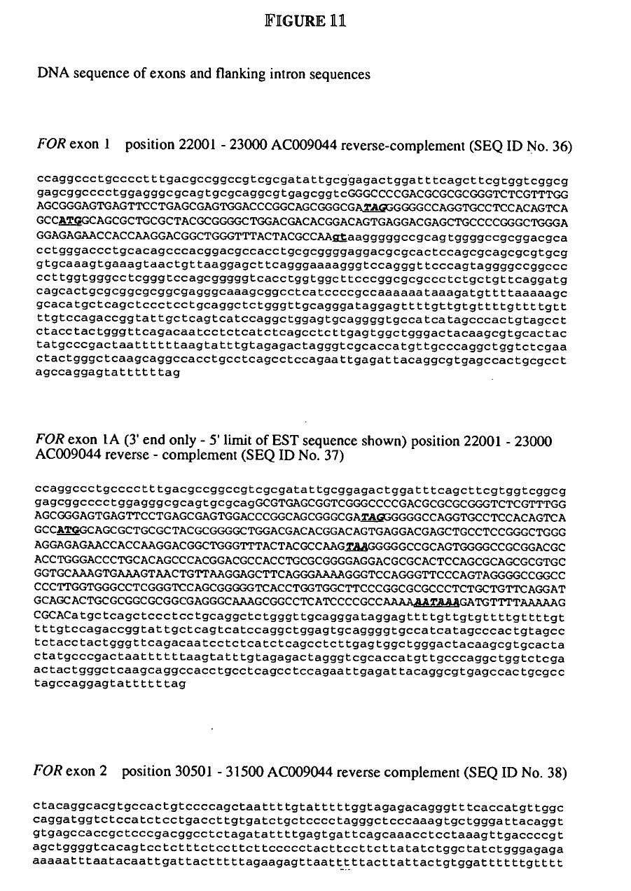

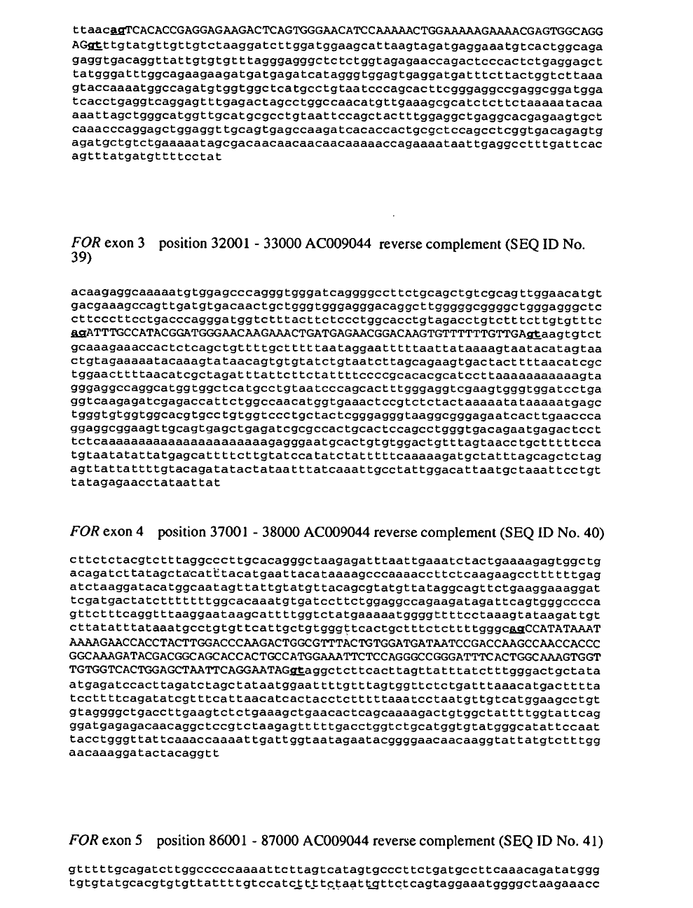

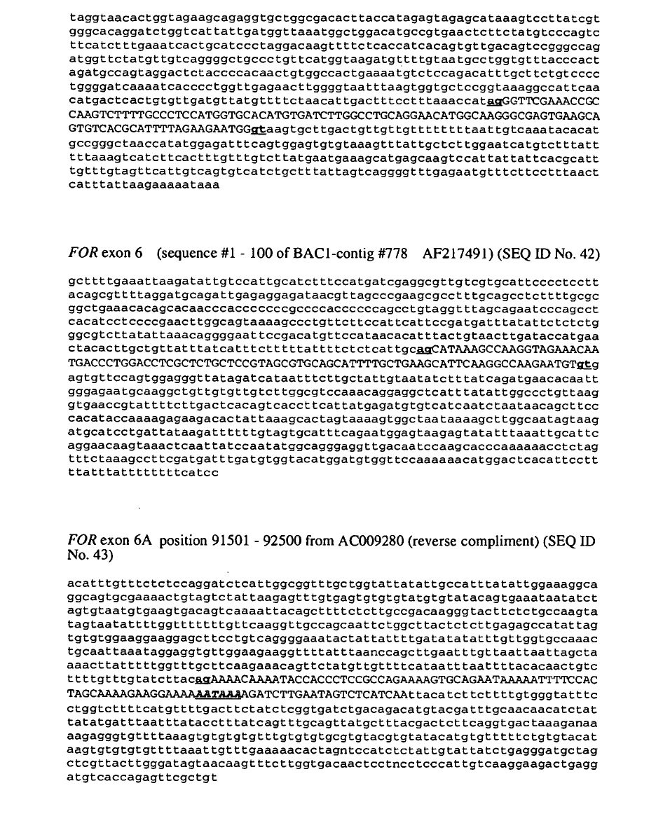

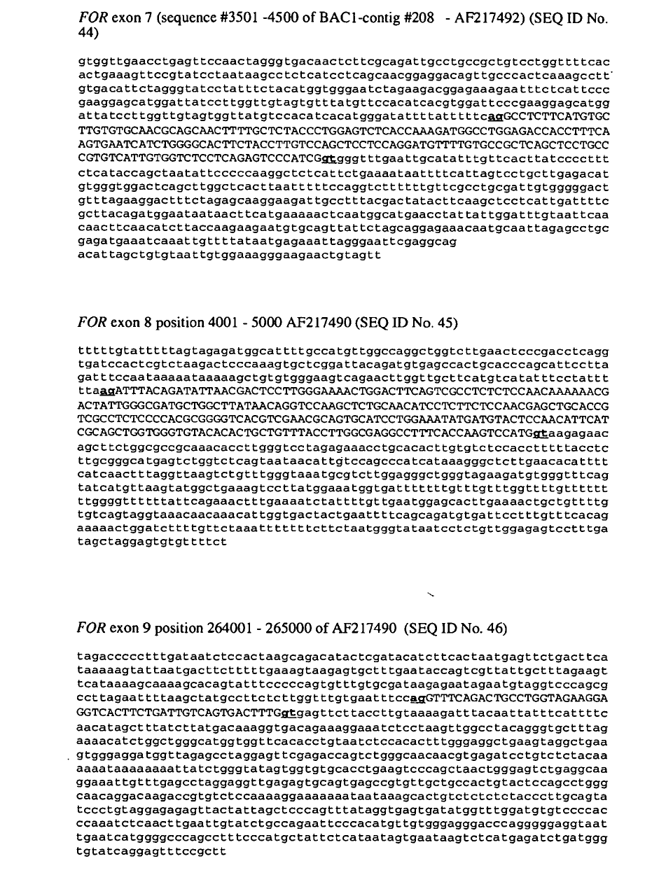

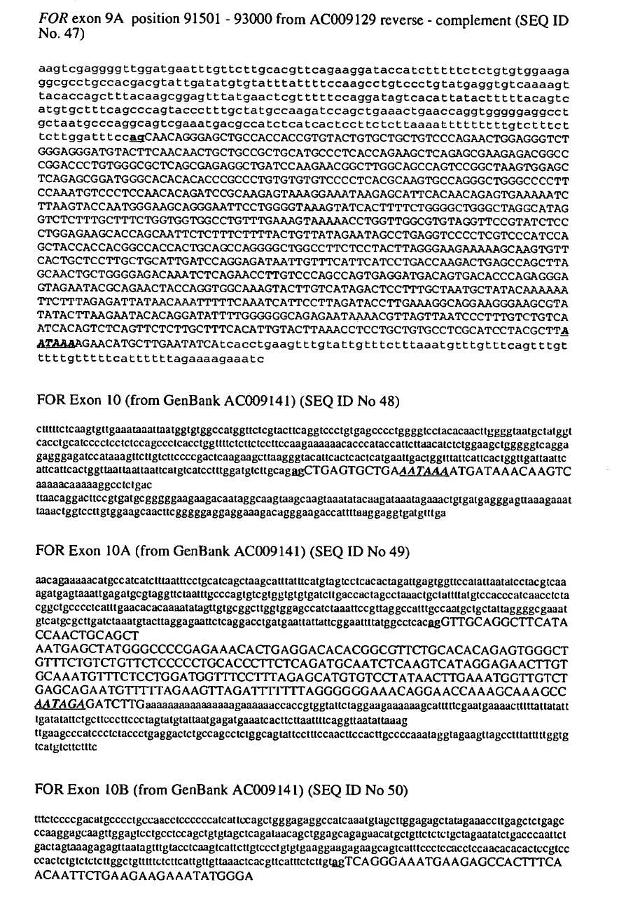

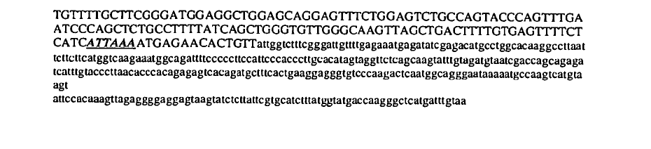

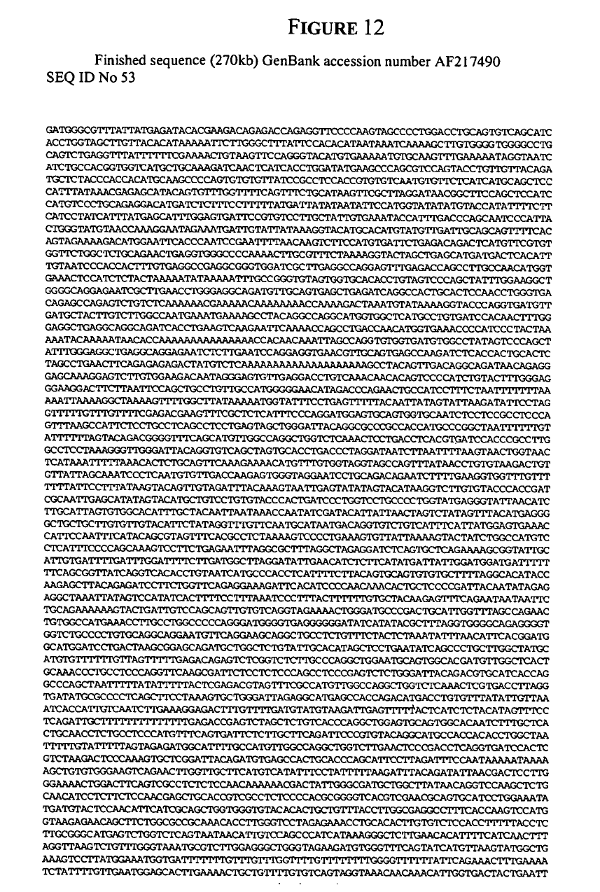

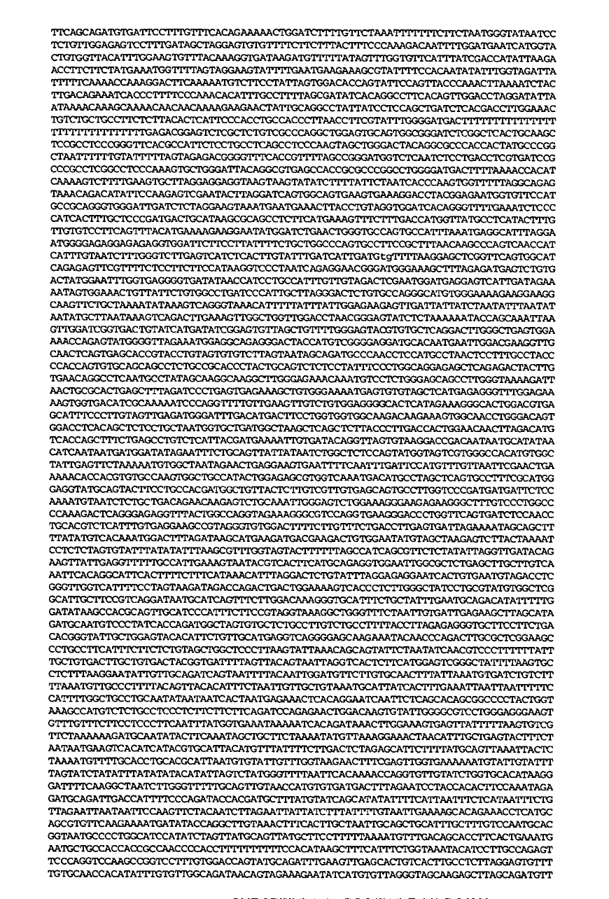

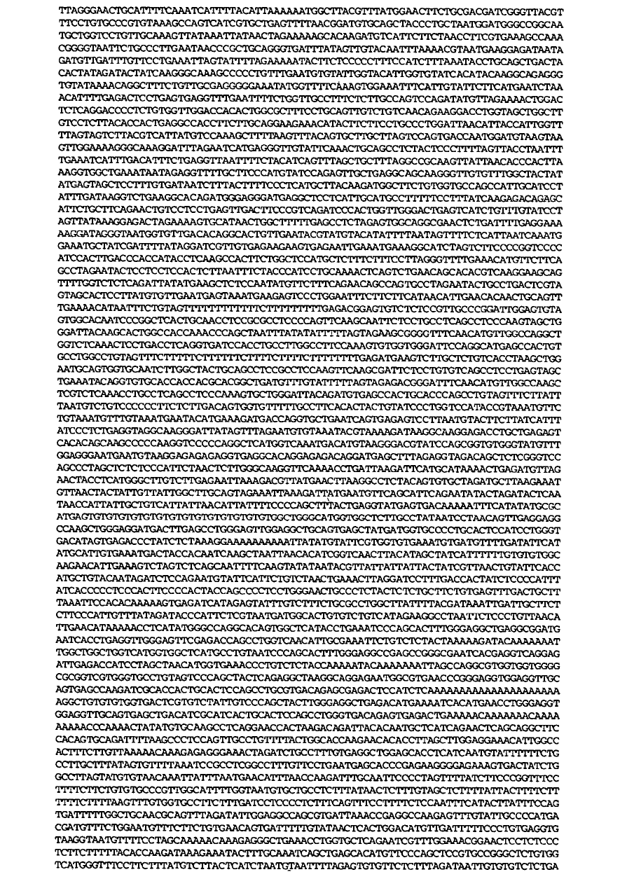

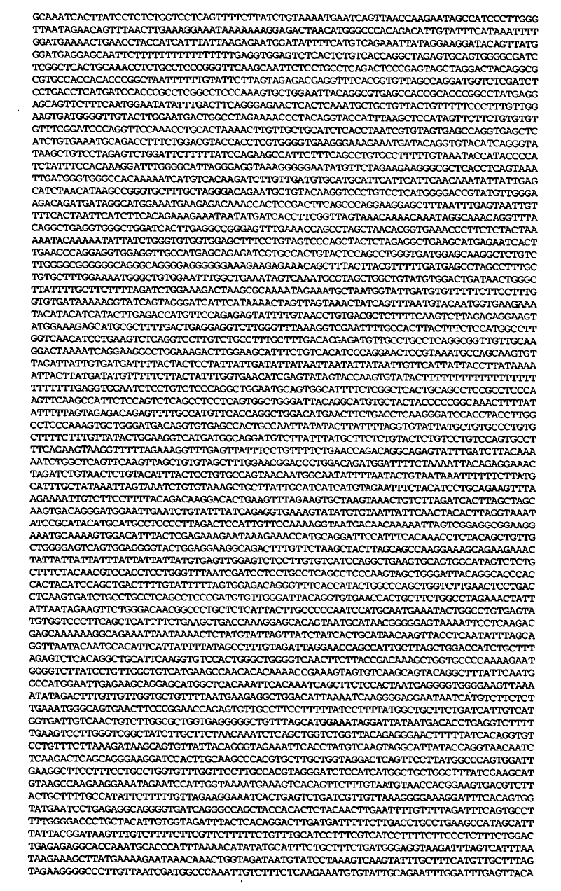

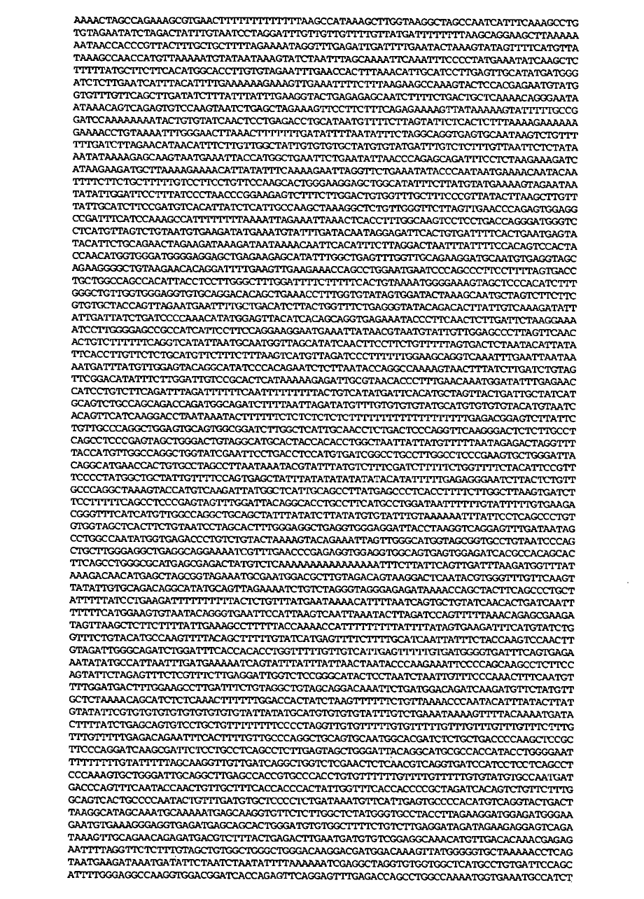

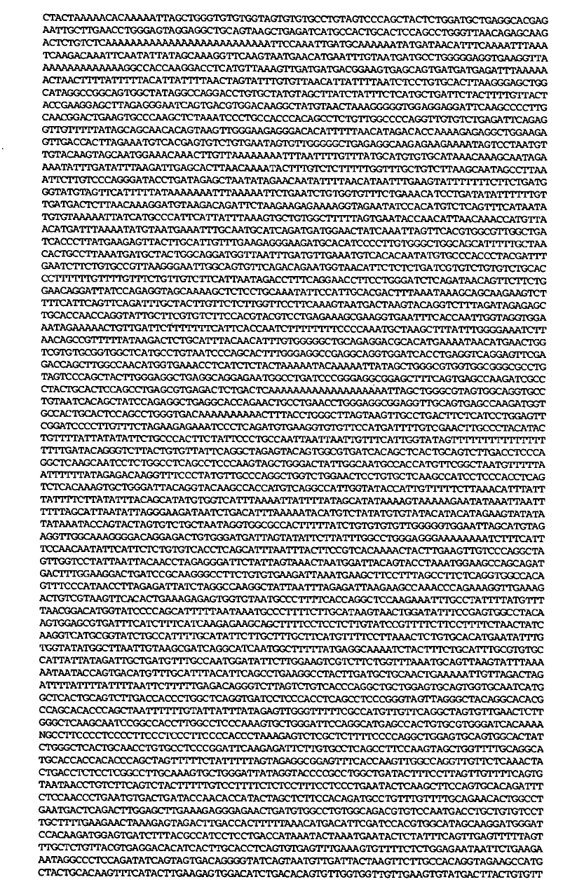

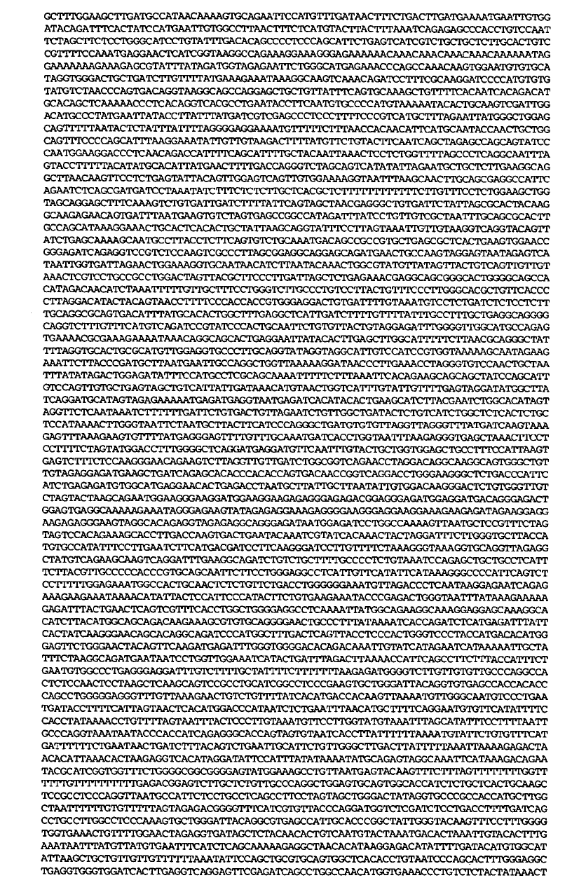

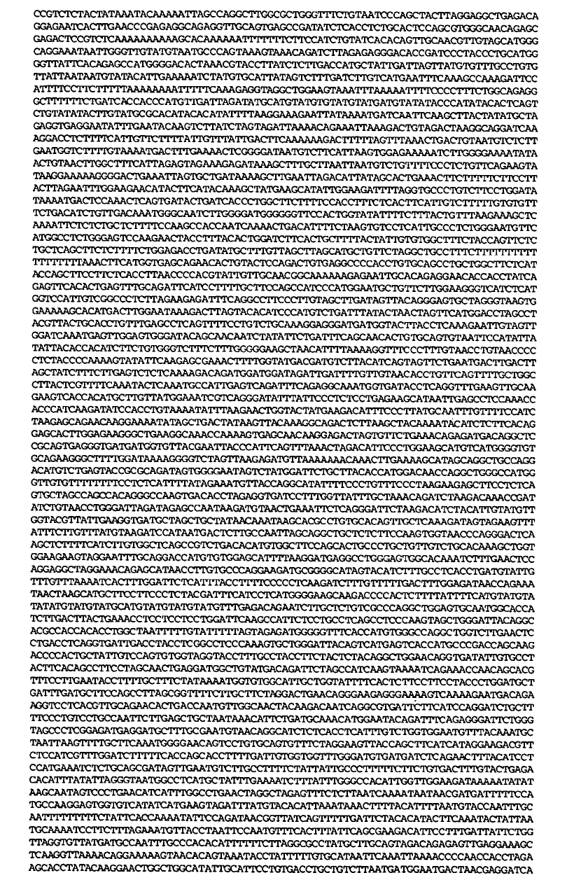

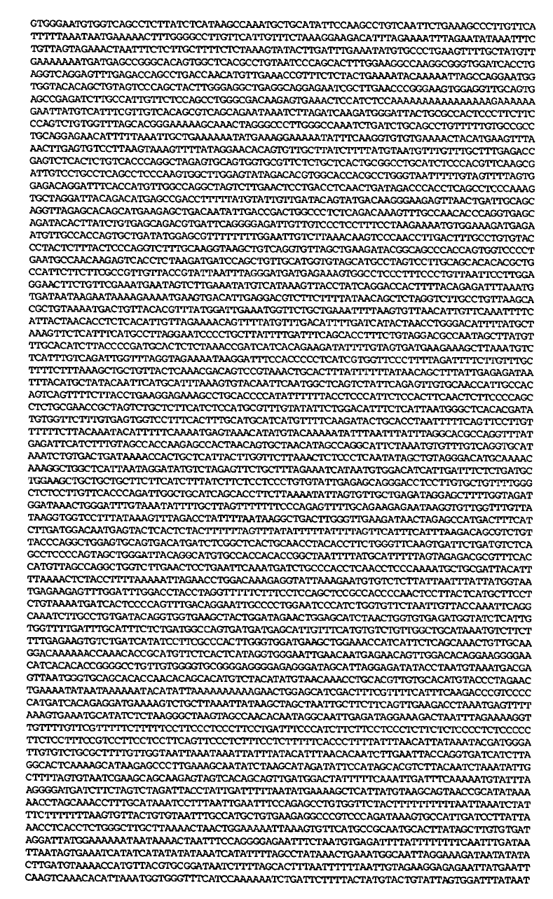

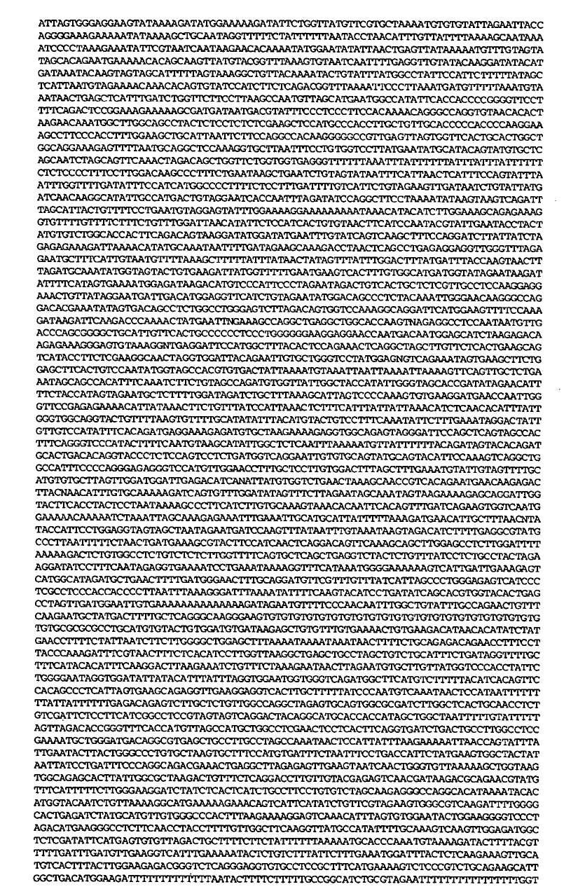

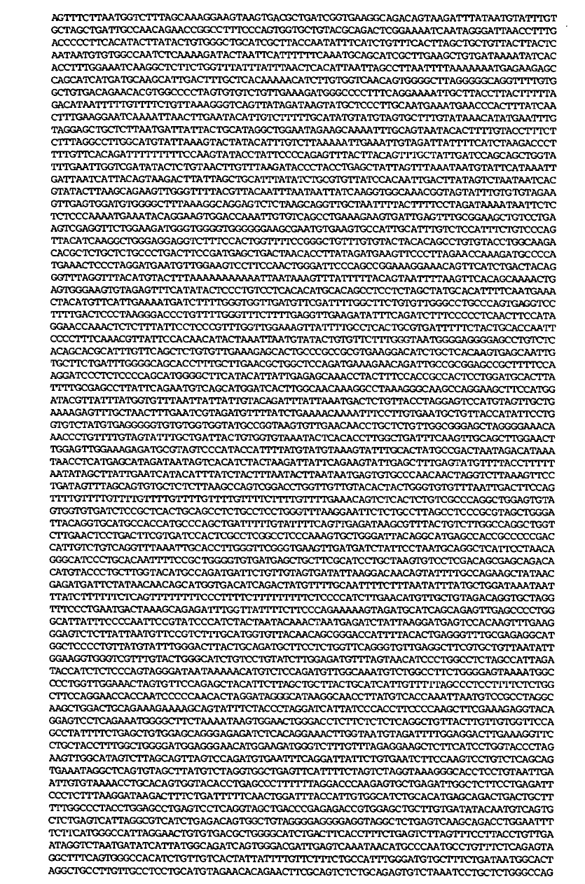

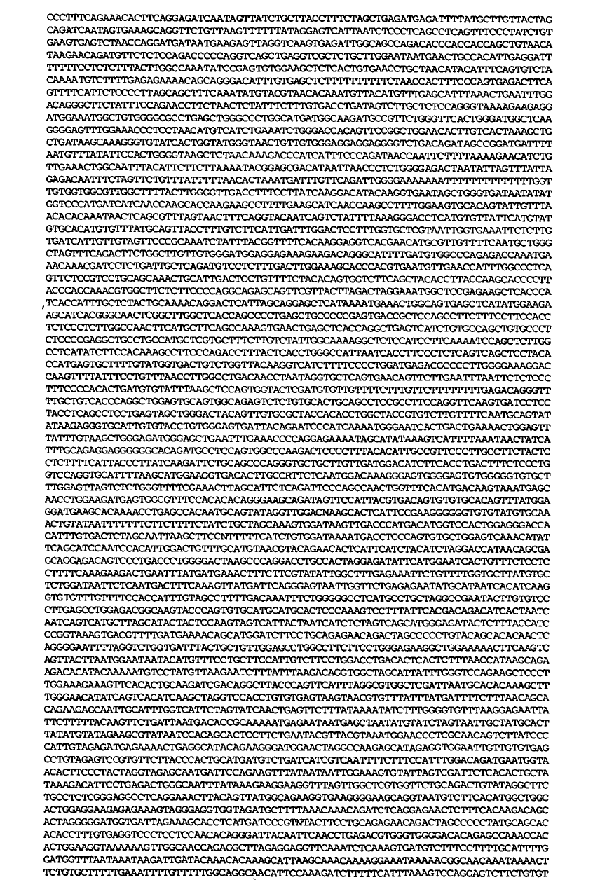

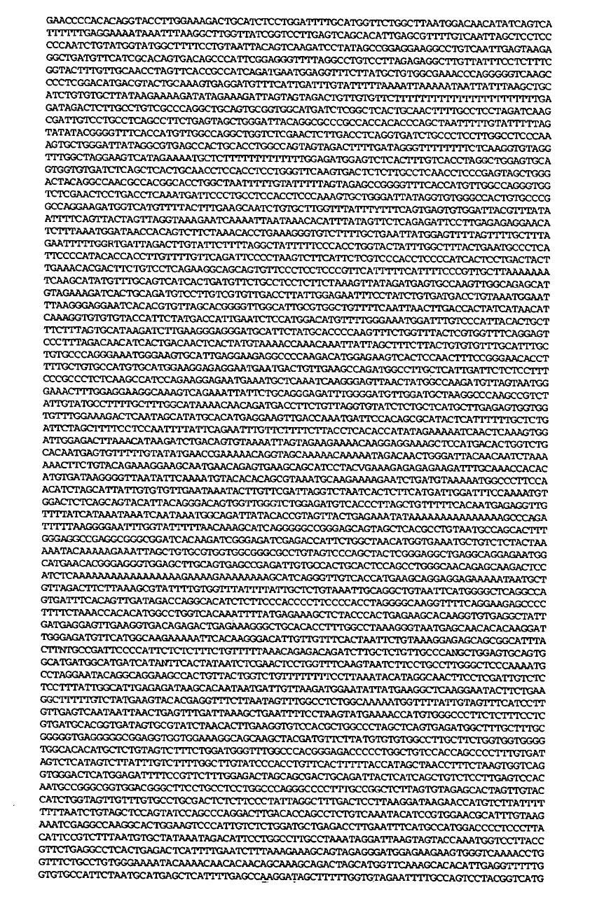

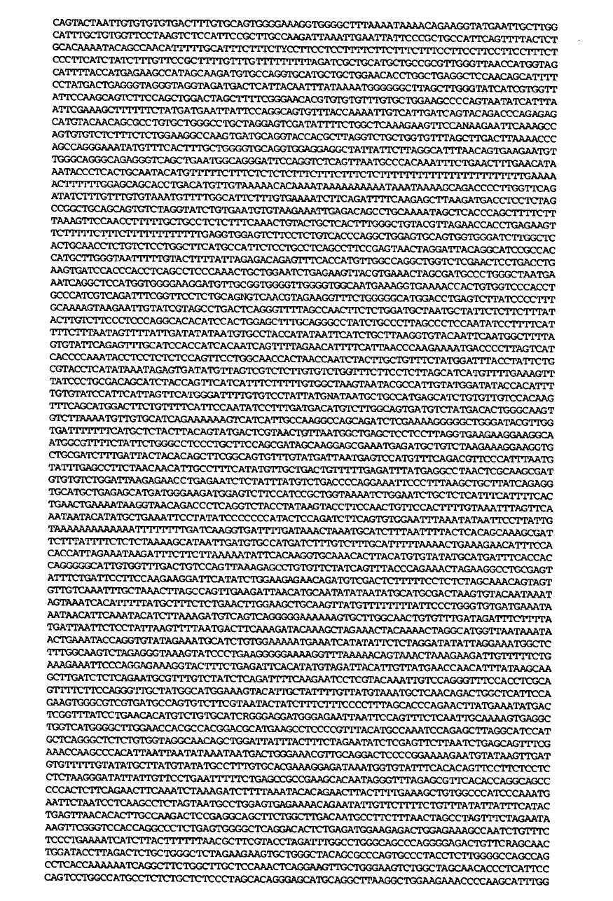

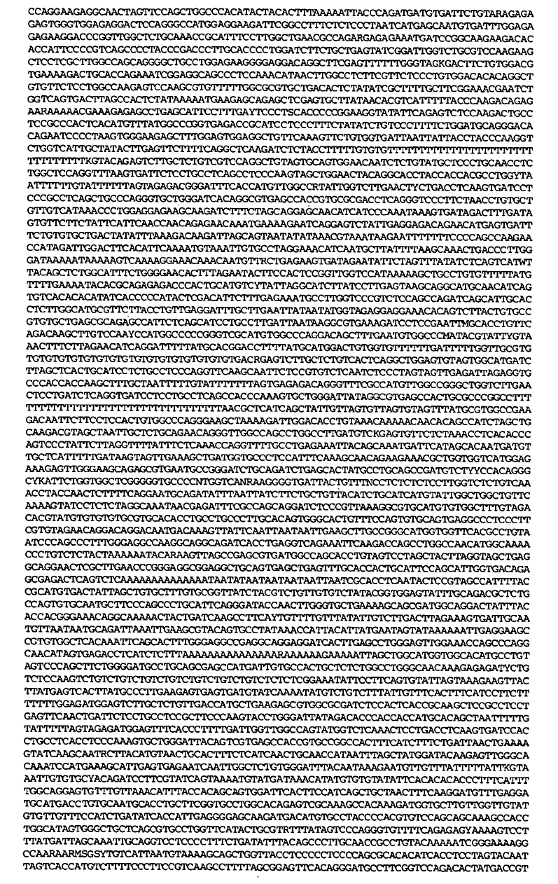

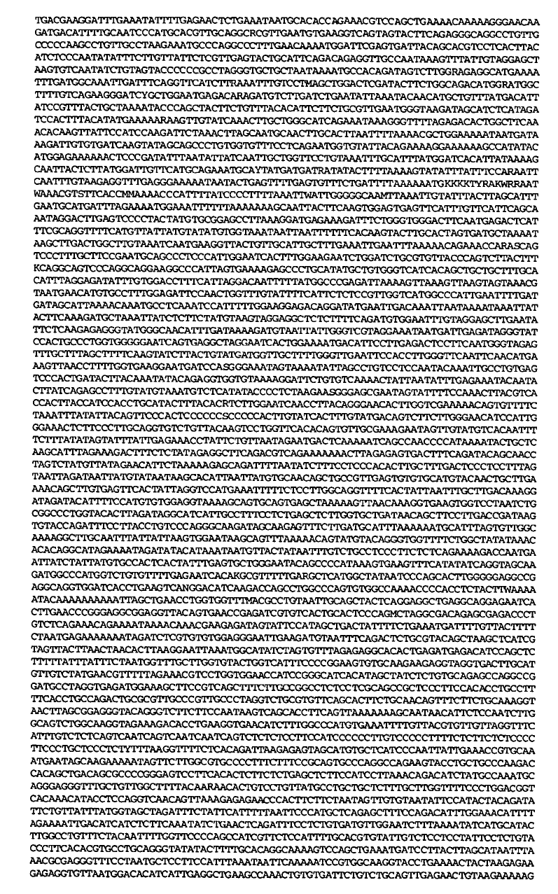

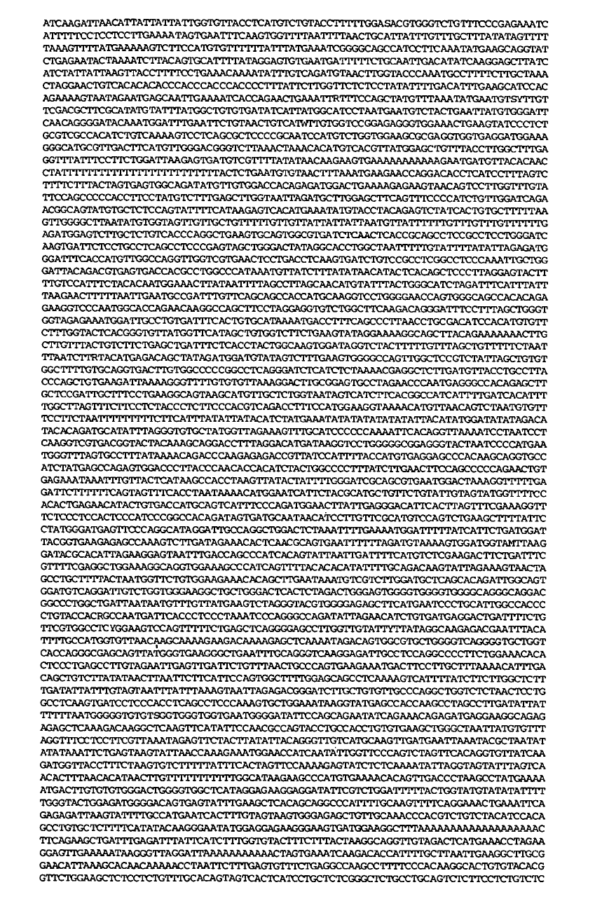

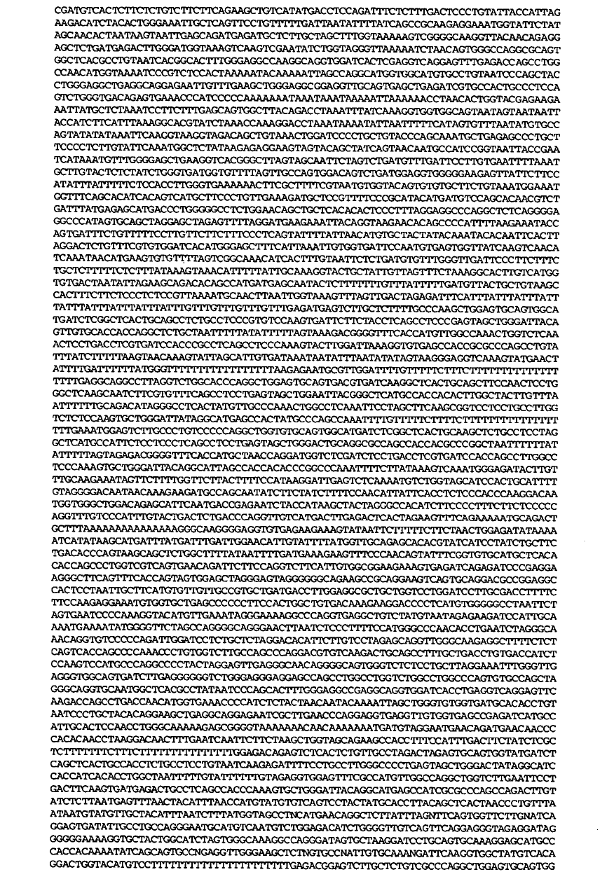

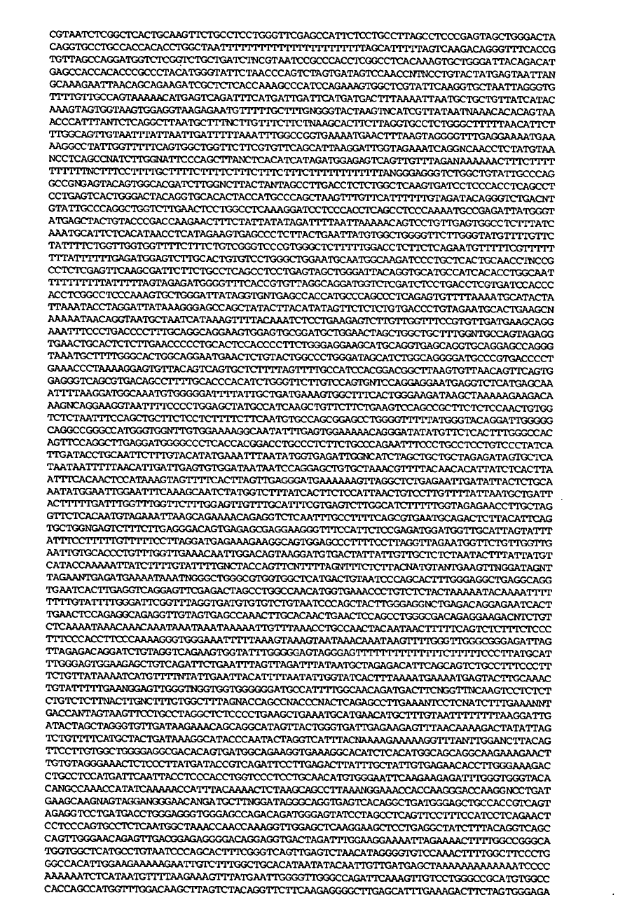

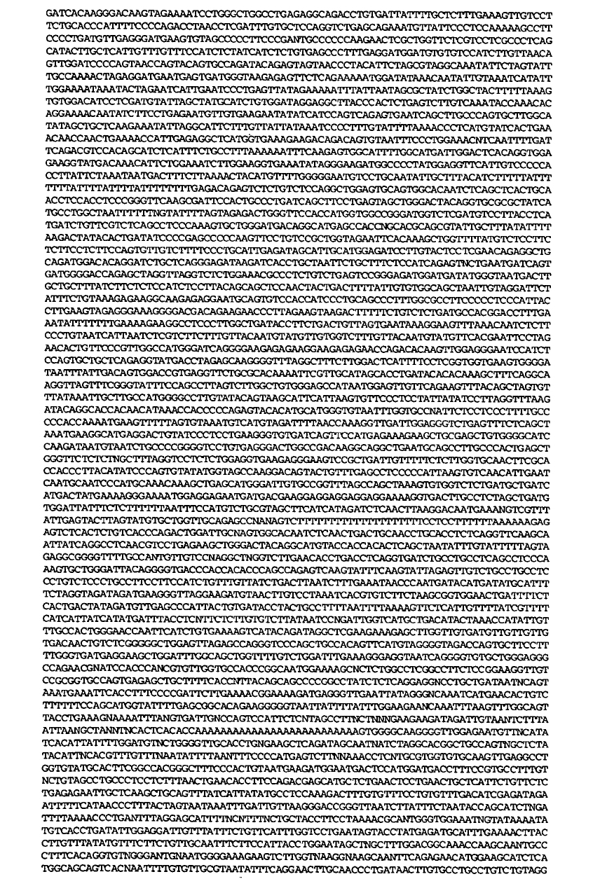

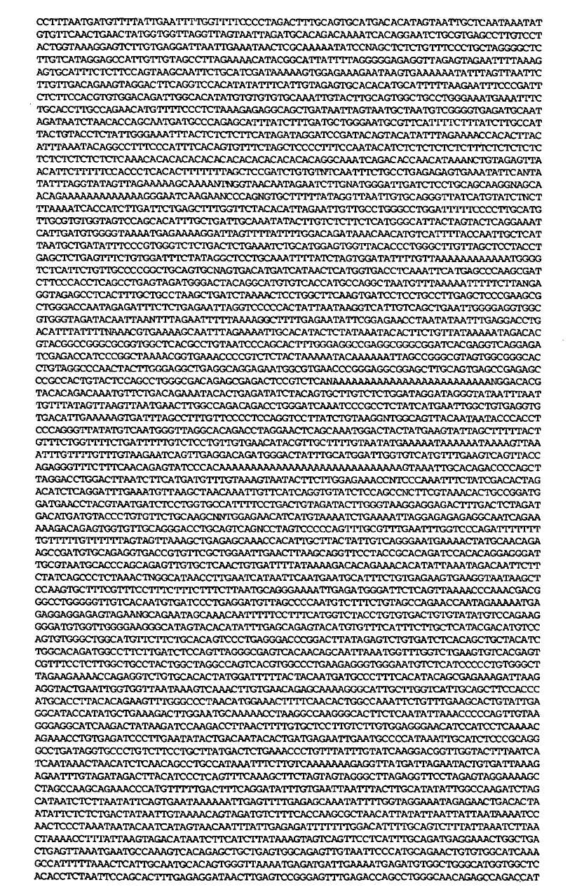

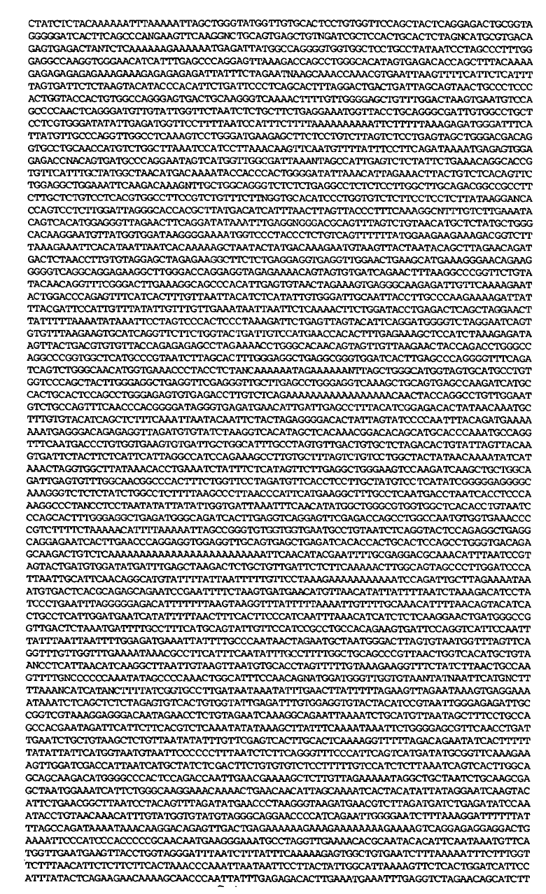

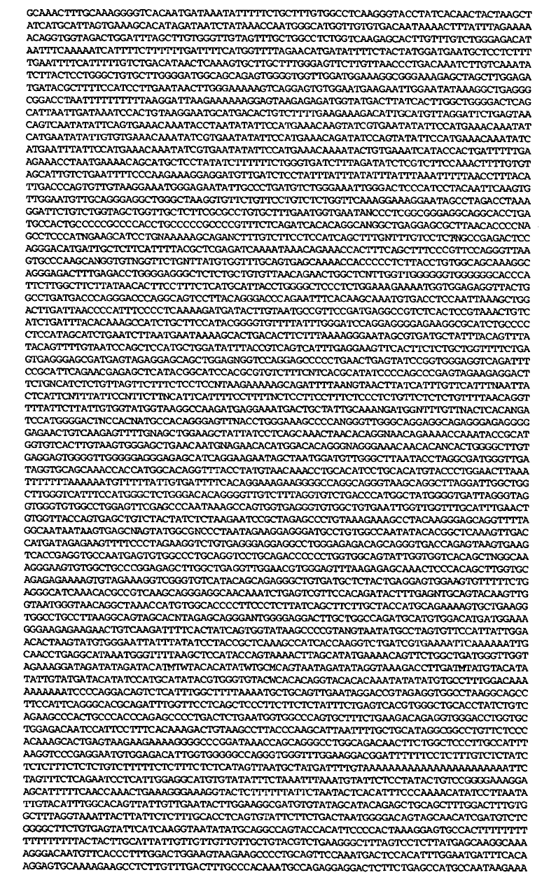

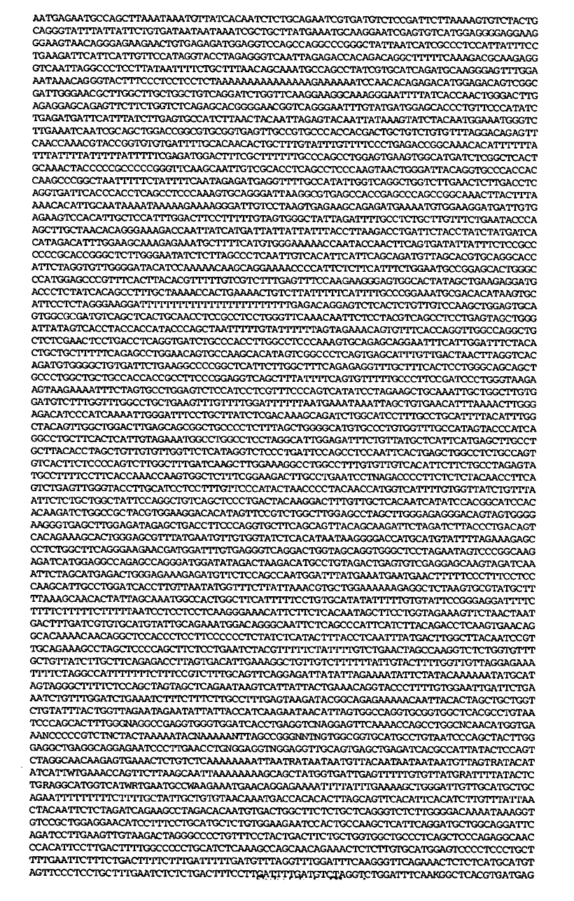

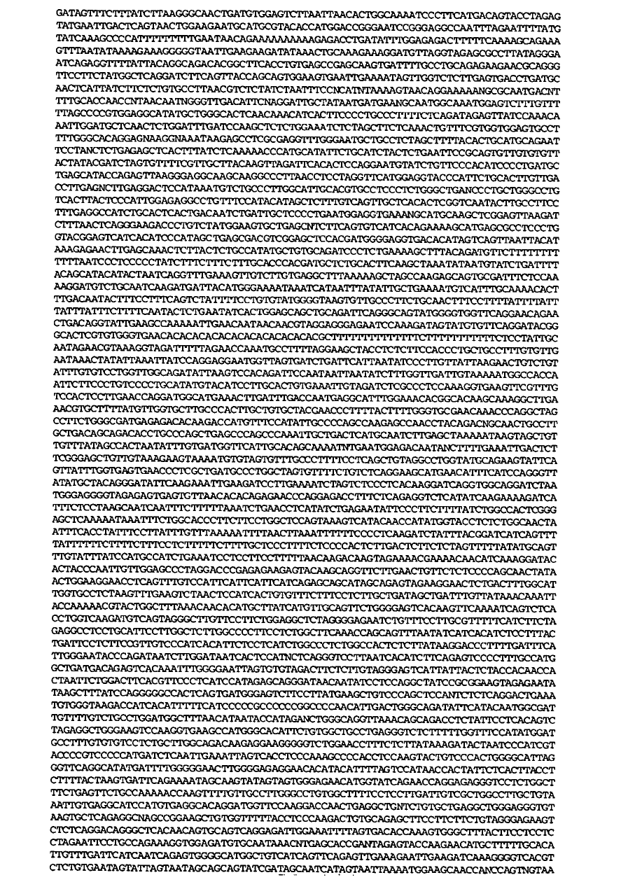

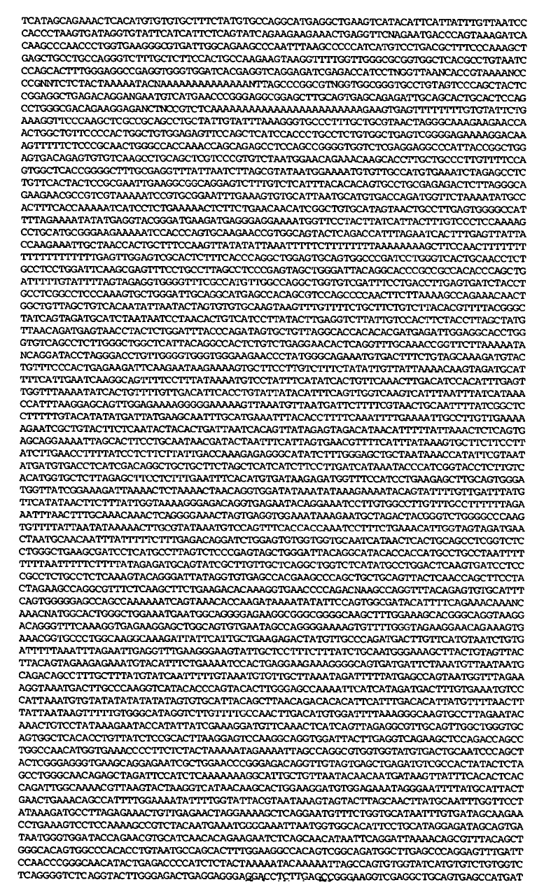

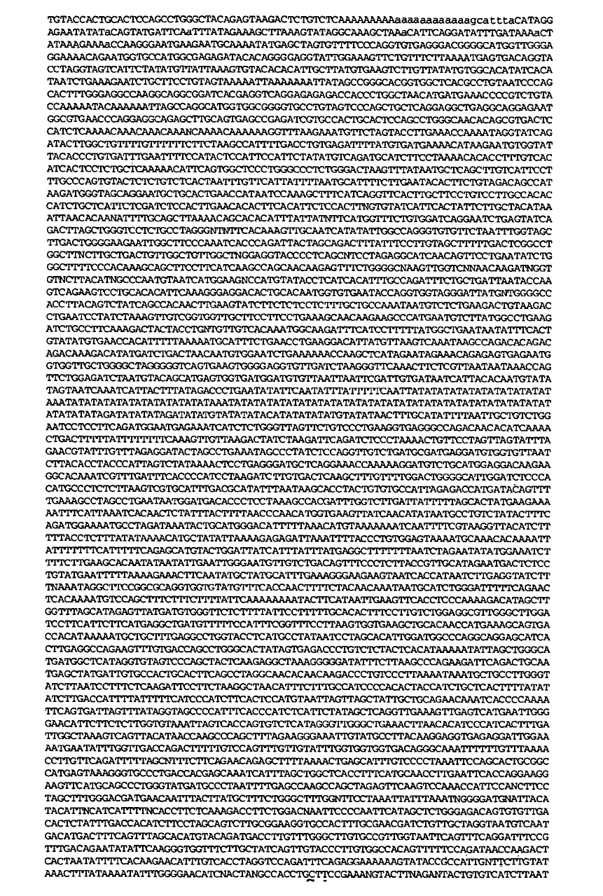

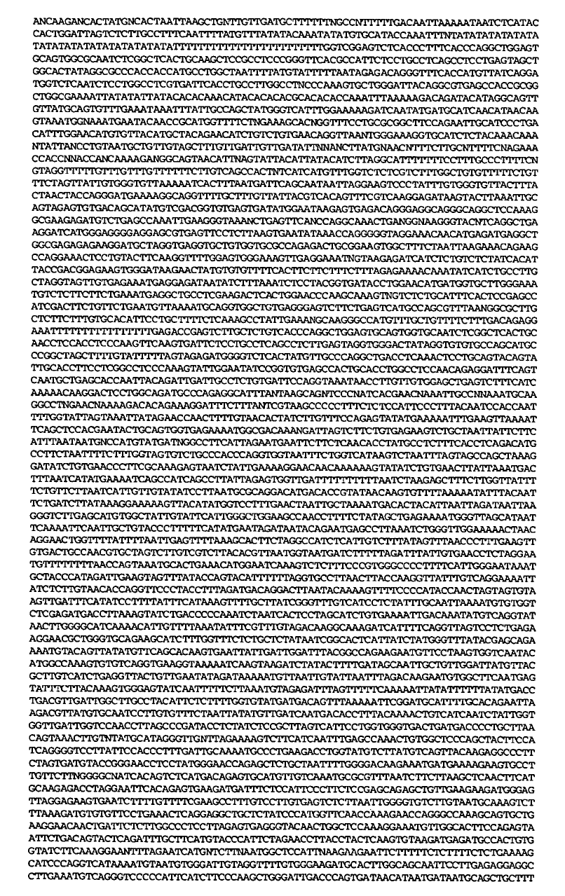

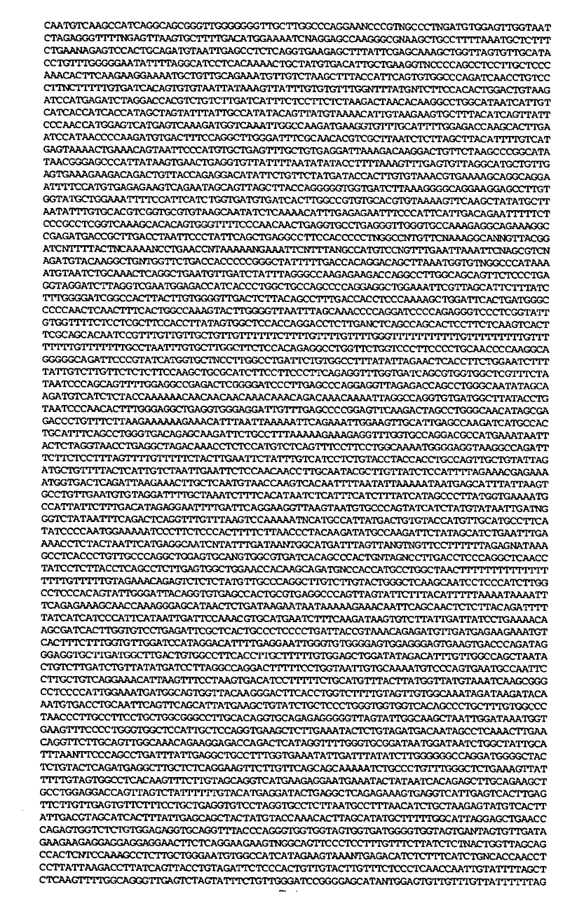

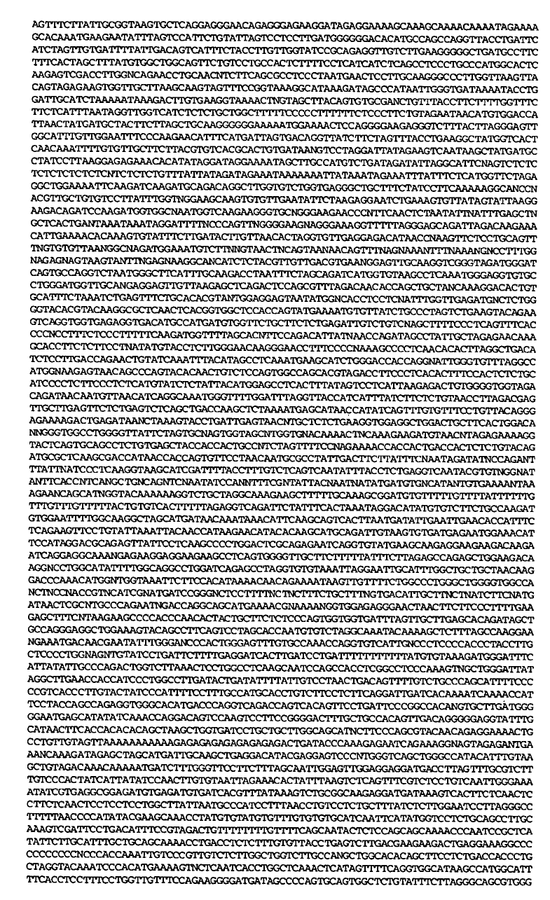

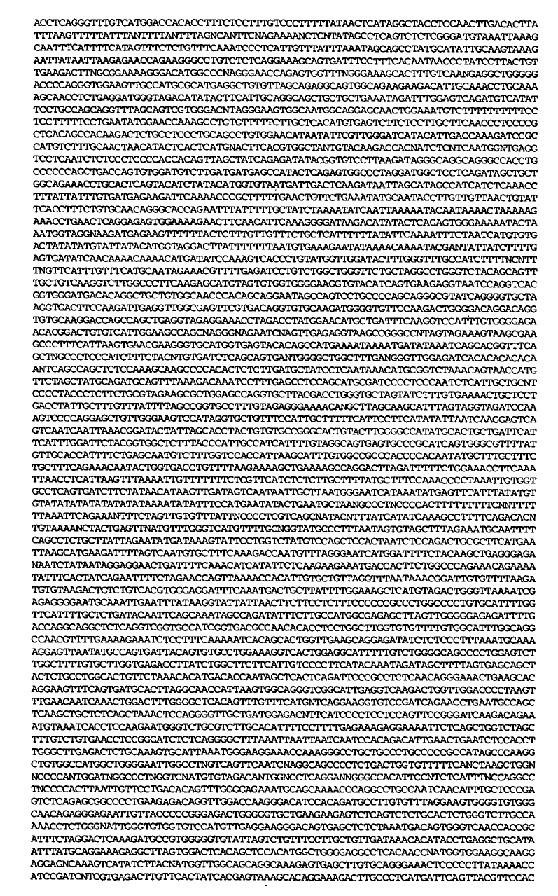

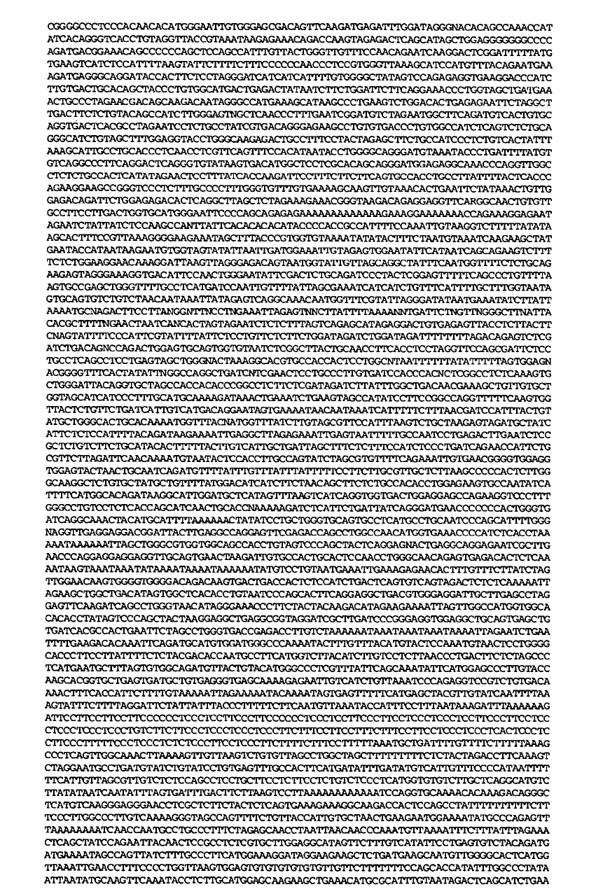

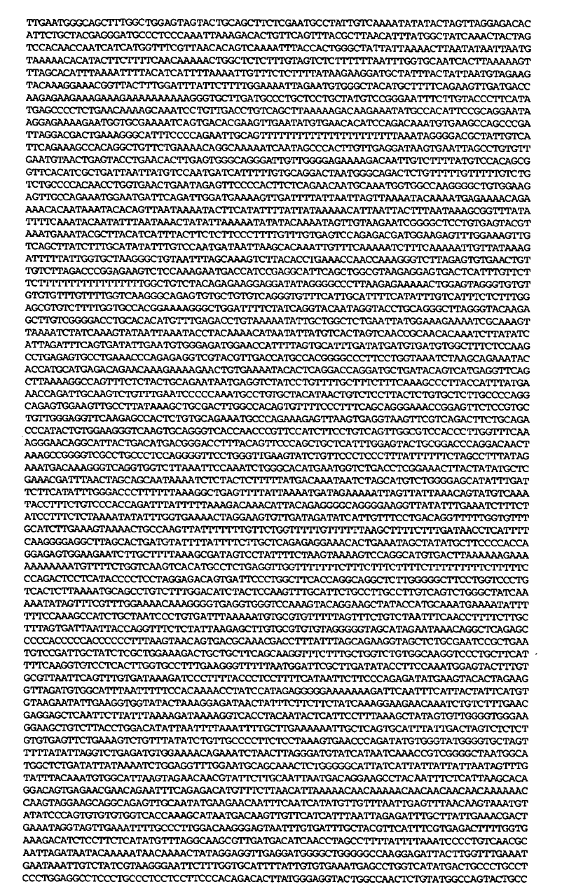

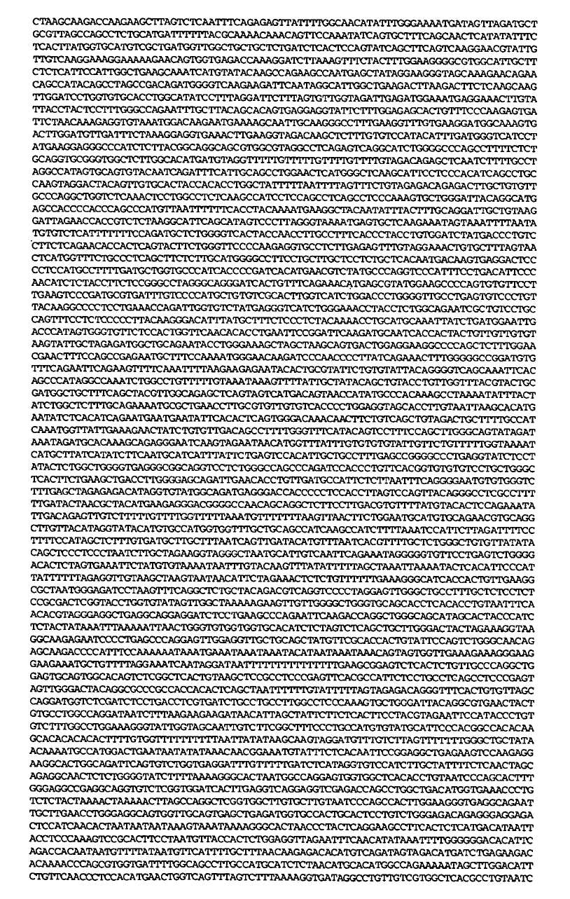

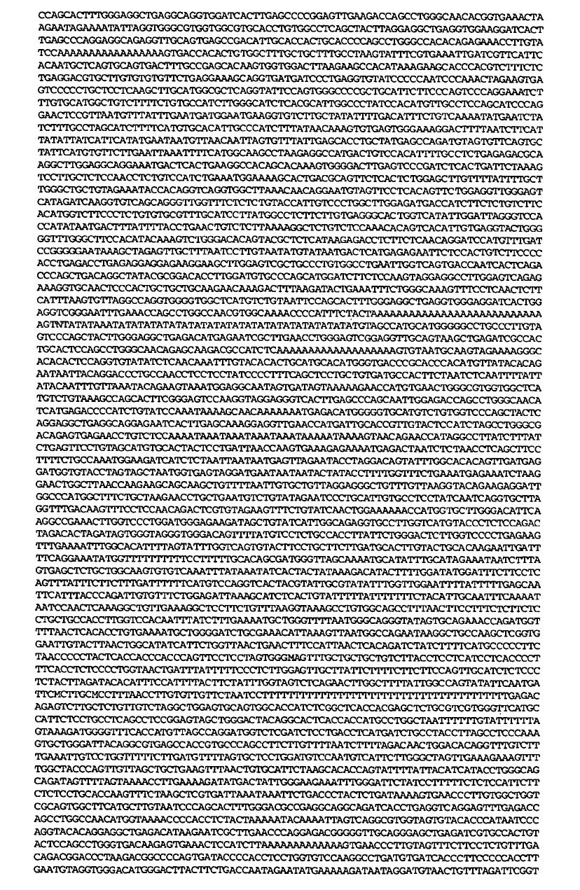

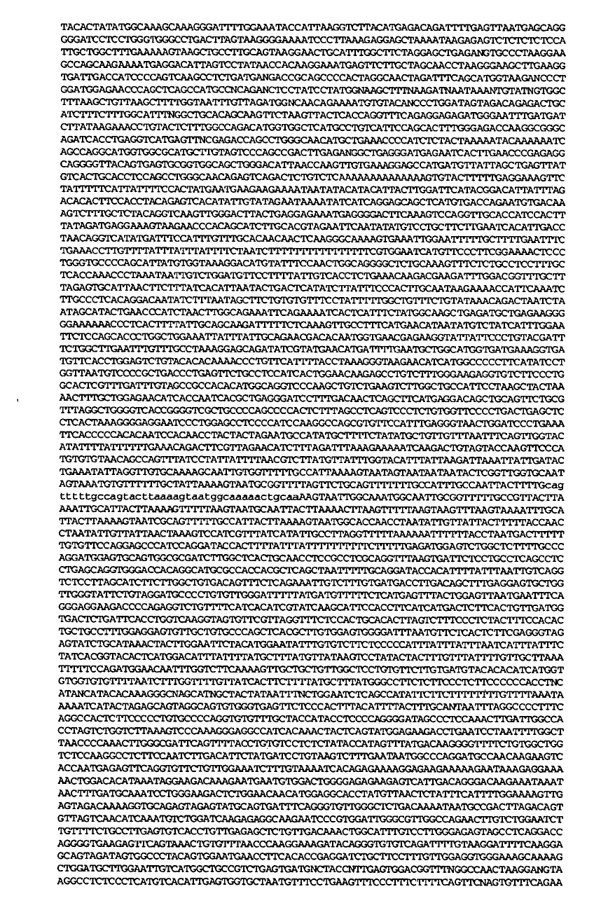

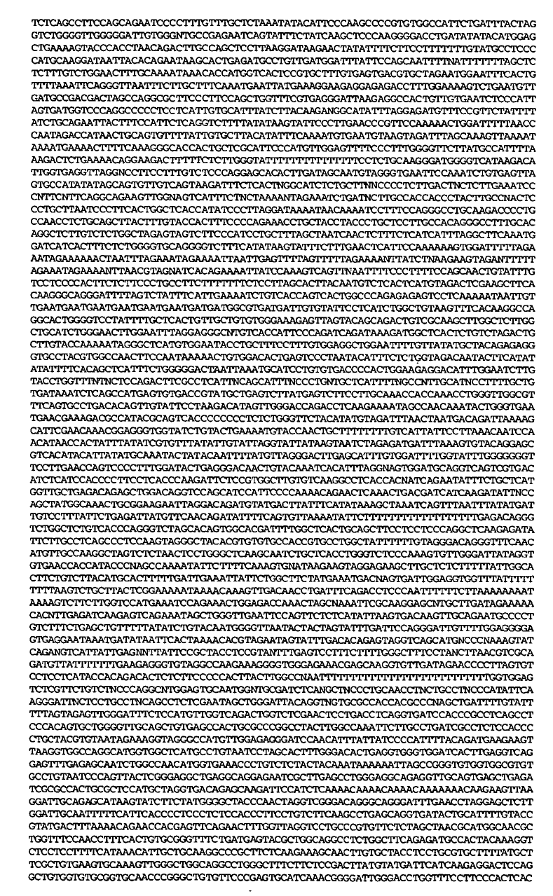

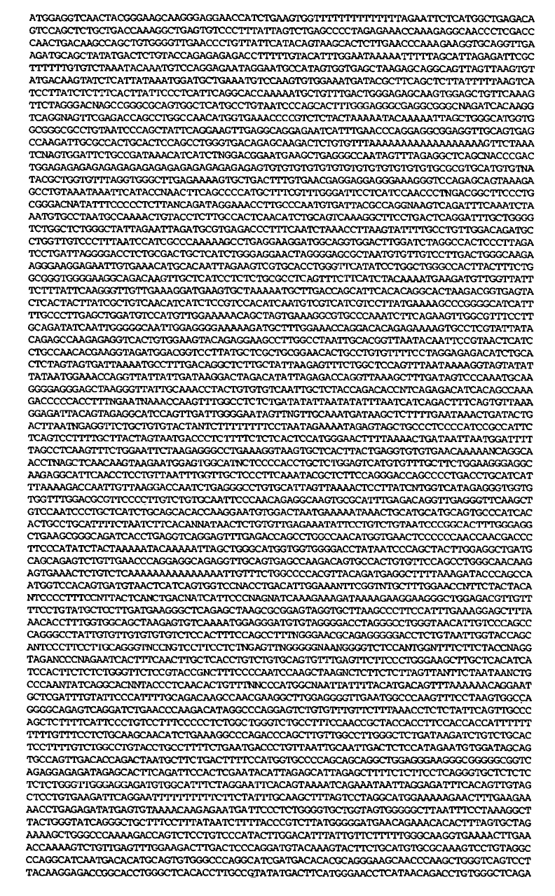

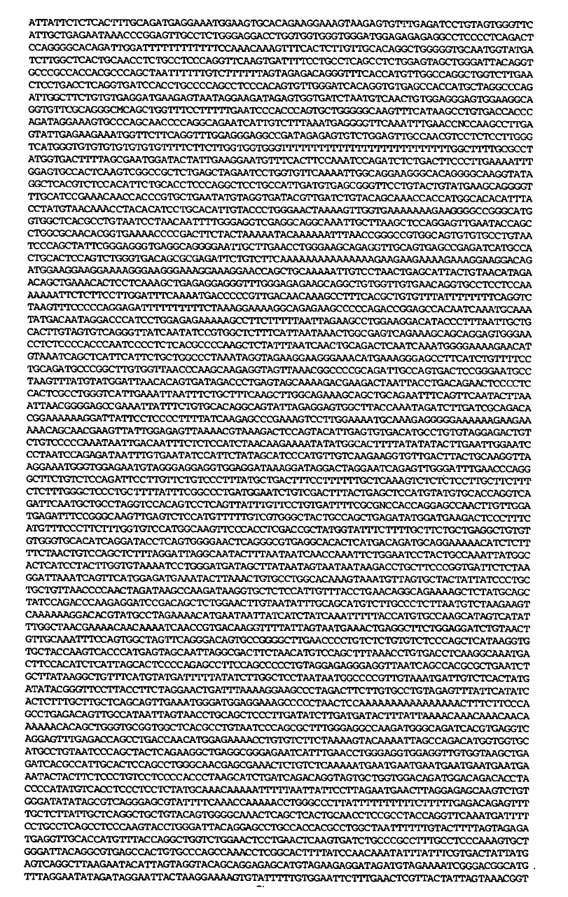

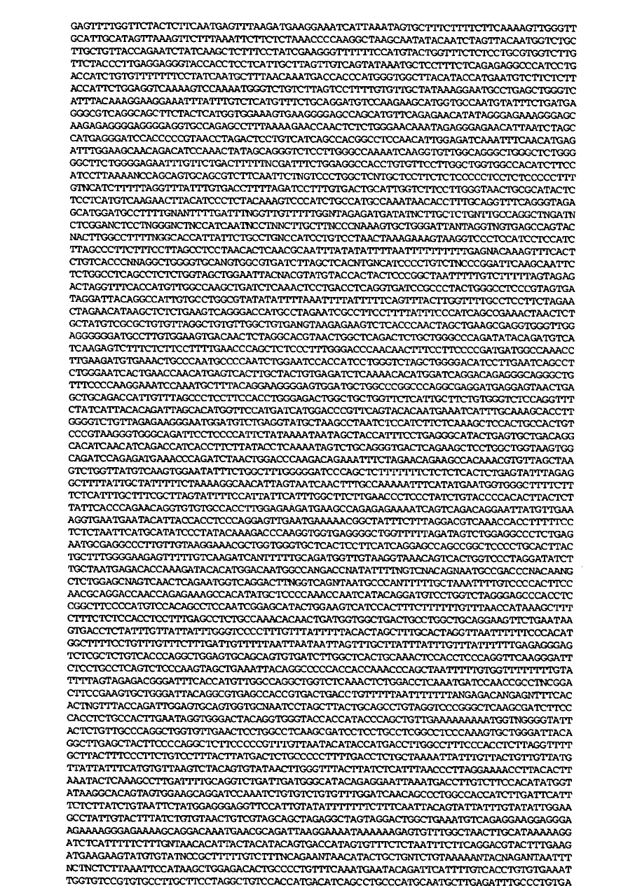

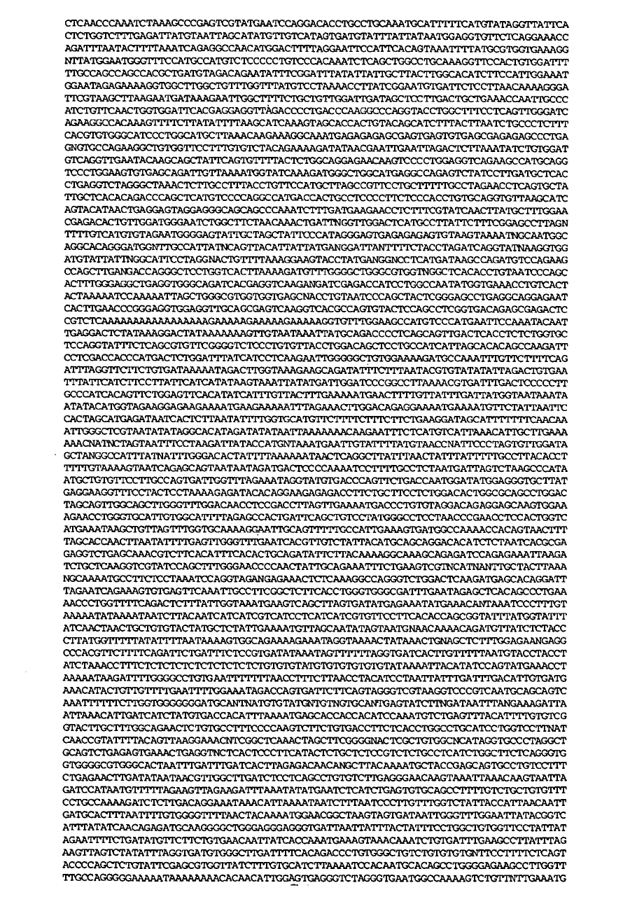

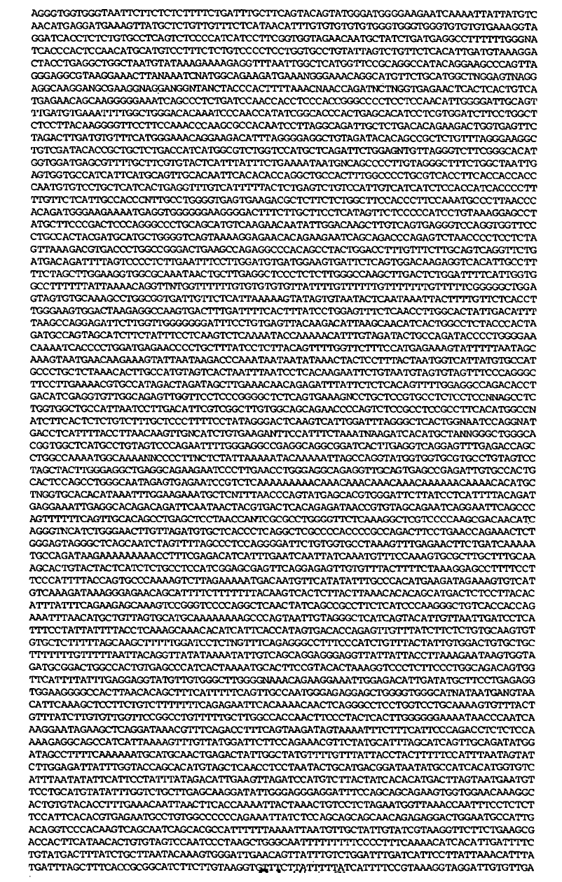

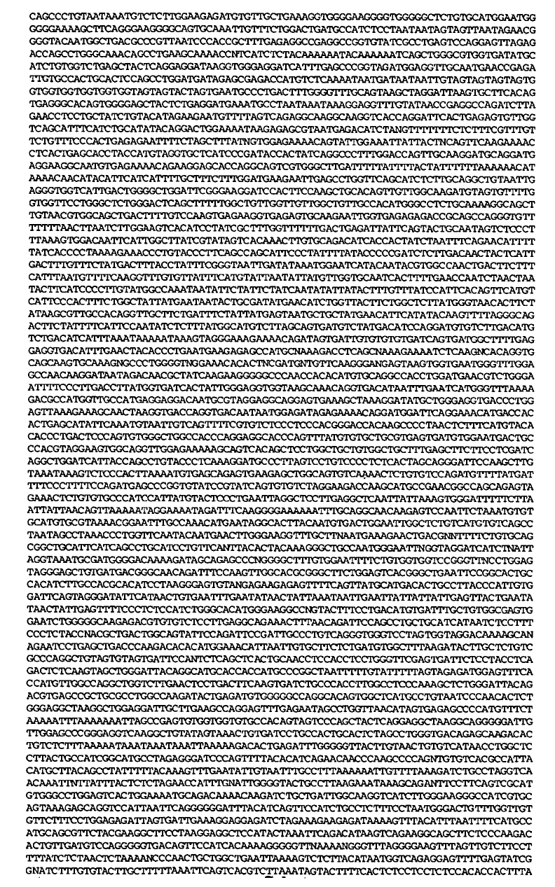

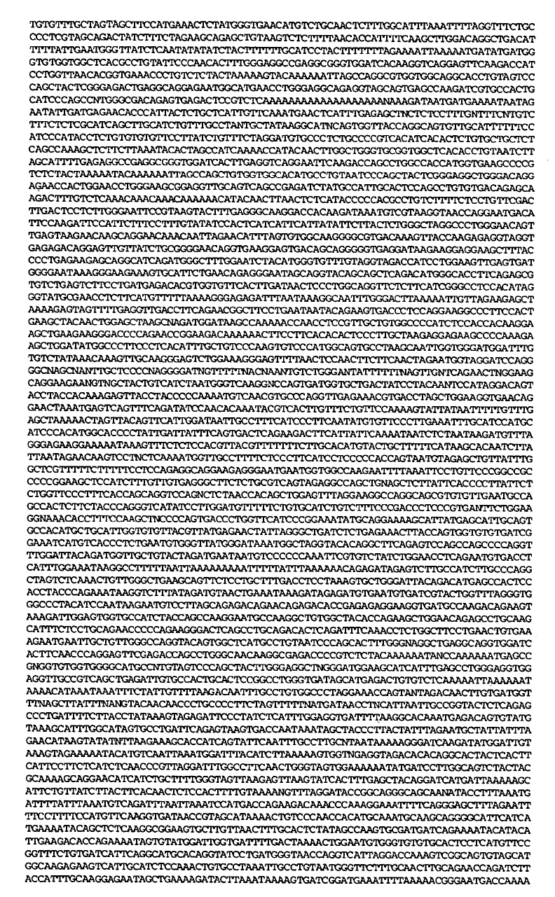

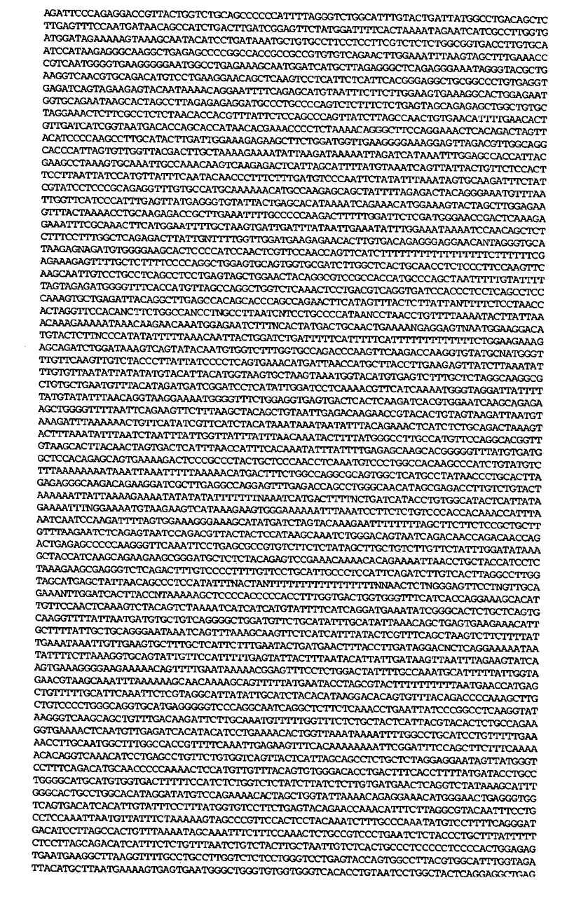

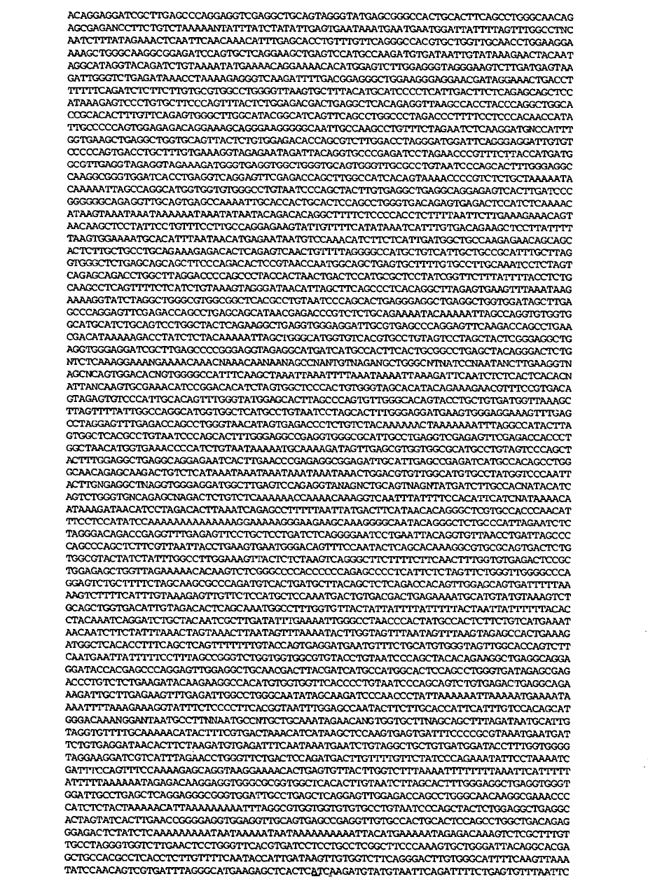

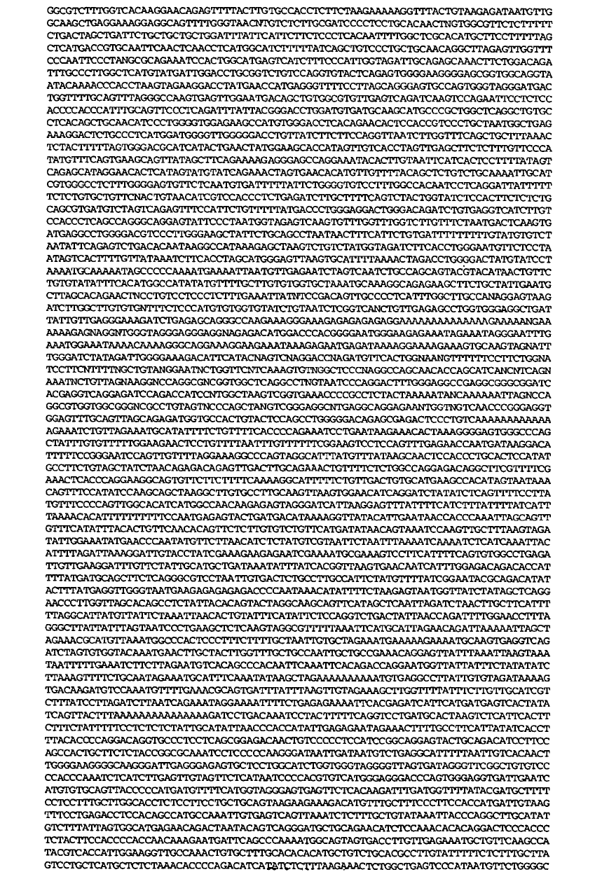

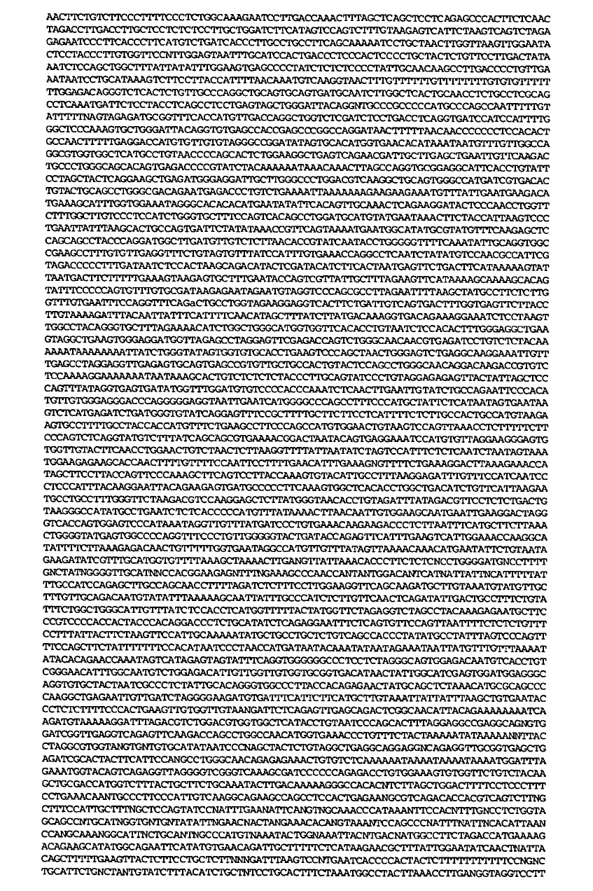

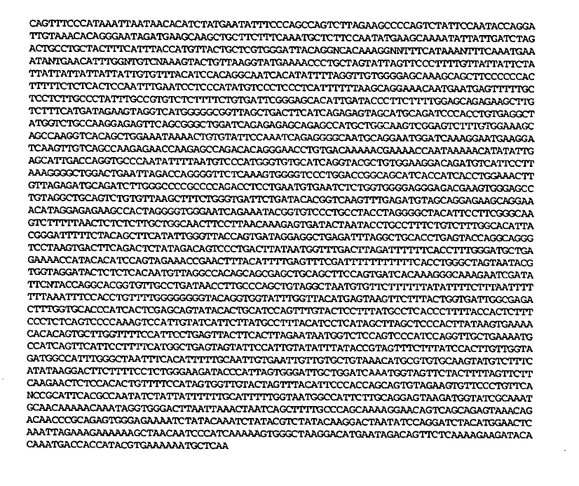

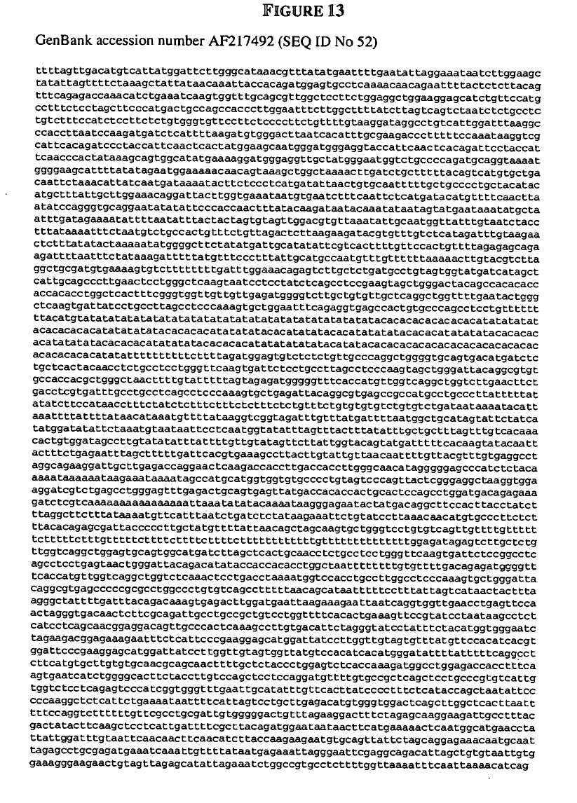

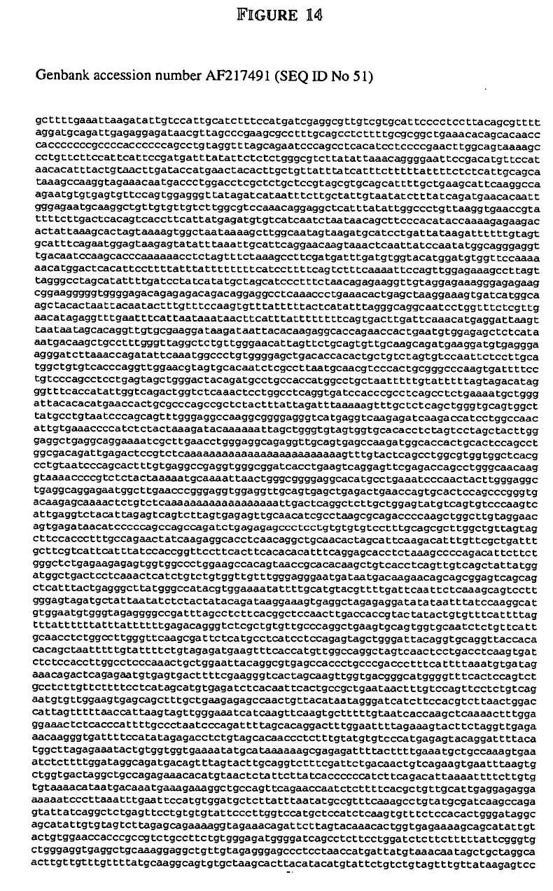

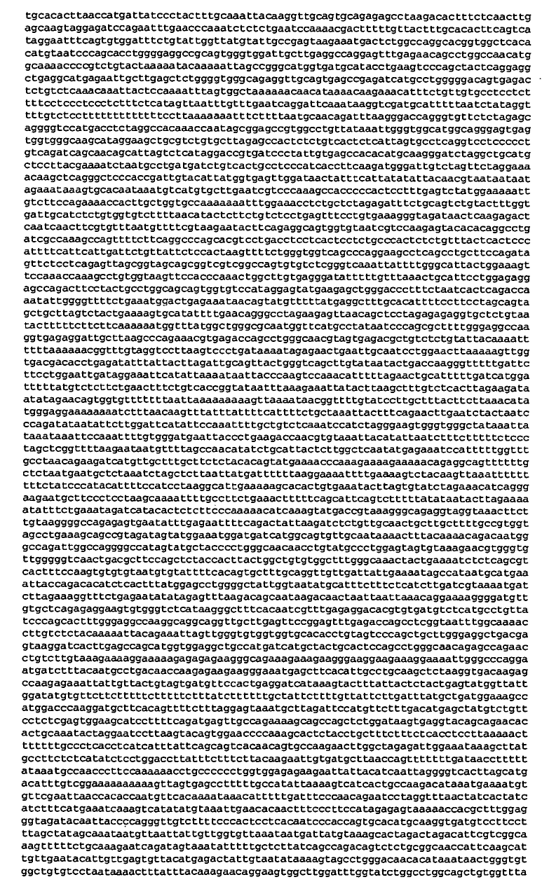

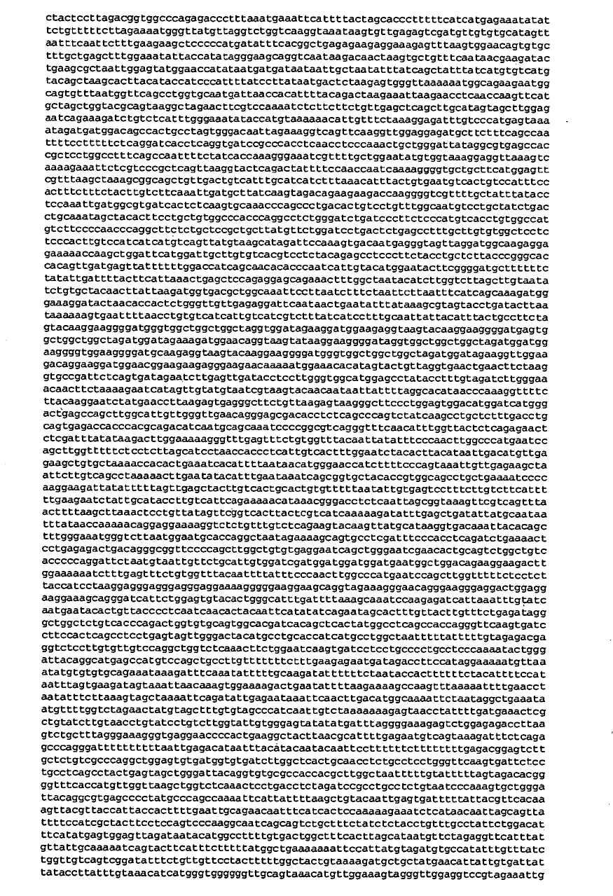

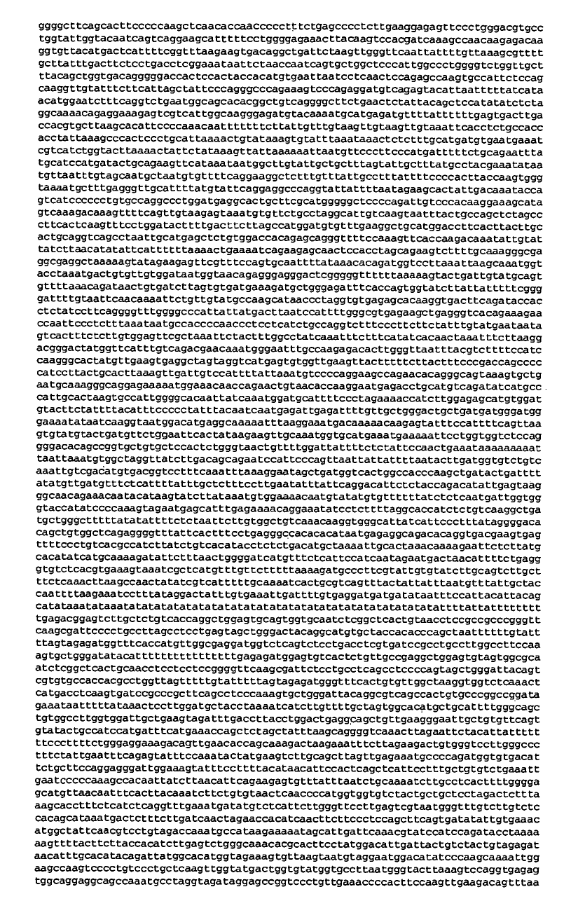

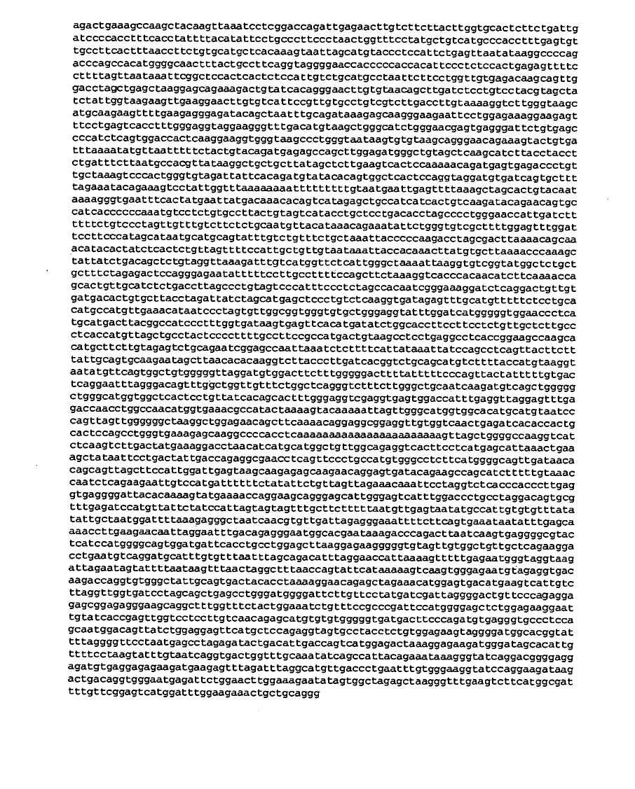

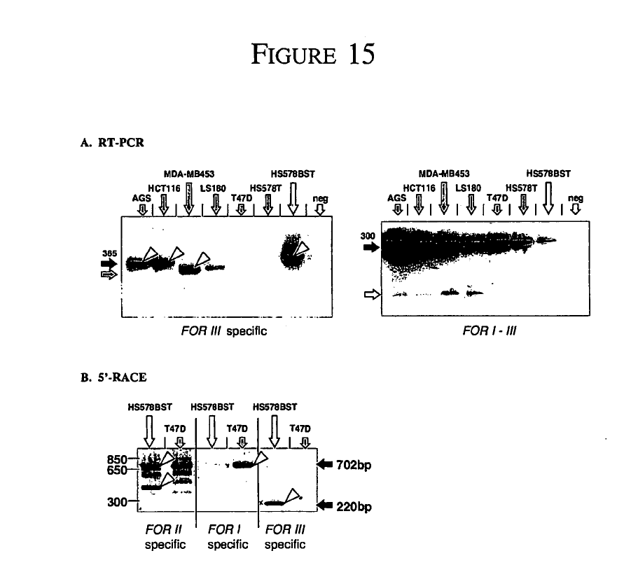

Nine DNA probes, ACH202 (D16S14), c311F2, c302A6 (D16S1075), c301F10 (D16S373), 16-87 (D16S181), c306D2, 16-08 (D16S162), c307A12 and CRI-0119 (D16S50) which had been physically mapped into the 16q23 region (30) were chosen for fluorescence in situ hybridisation (FISH) against FRA16D expressing chromosomes. Four of these markers mapped within the same somatic cell hybrid breakpoint interval defined by the cell lines CY113(P) and CY121 (30). One of these, c306D2 mapped proximal to FRA16D by FISH while the others, c307A12, CRI-0119 and 16-08 mapped distal to FRA16D. These probes were therefore used as starting points to isolate a contig of cloned DNA spanning FRA16D. In the Los Alamos National Laboratory database (www-ls.lanl.gov) an STS sequence from c306D2 was found within the CEPH YACs My903D9, My912D2 and My933H2 while an STS in c307A12 was found in My891F3 and My972D3. These YACs were obtained from CEPH and the prepared DNA subjected to Pst I digestion, Southern blotted and probed with 16-08, 16-87, CRI-0119, c306D2 and c307A12 in succession in order to confirm their content. In addition a search of the Whitehead Institute database (www-genome.wi.mit.edu) revealed that the two sets of YACs were joined into a contig by the YACs My801B6, My845D9 and My944D8. Each of these YACs was used as template DNA to assess STS content (D16S518, Afma336yg9, WI2755, STSG-10102 and D16S3029) and subjected to FISH to assess position with respect to FRA16D (FIG. 1B).

Additional Probes, STSs and BACs from the FRA16D Region

Additional probes were generated from the YAC 801B6 by subcloning Pst I digests of YAC DNA and screening with total human DNA as probe. These subclones were digested with Hinc II to identify and isolate non-repetitive DNA fragments as probes. This generated markers H13m, H22s, H23m, H29m and H40m. Genome System Inc. BAC library filters were screened with the probes D16S518, Afma336yg9, WI-2755, STSG-10102, H22s, H29M and D16S3029 and nine BAC clones including 379C2, 325M3 and 353B15 were identified. An additional STS, named 2AS, was established by ‘bubble’ PCR from the end-fragment of BAC 353B15 and was isolated as described by Gecz et al (31). Briefly, the BAC DNA was digested with Alu I and ligated to the annealed bubble linkers. The final PCR was carried out with a combination of Not I-A bubble primer and Sp6-promoter primer as described except an annealing temperature of 55° C. was used. These STSs and hybridisation probes were used to establish restriction maps of the YAC My801B6 and the BACs (FIG. 2A).

Subcloning and Contig Assembly

The YAC My801B6 and the BAC 325M3 were used as DNA templates for establishing lambda subclone libraries in lGEM 11 or lGEM 12 vectors (Promega) according to the supplier's protocol. My801B6 and 325M3 appeared to have intact human DNA inserts, based on comparative pulsed field gel mapping of the YACs and BACs across the region (data not shown).

Fluorescence In Situ Hybridisation

FRA 16D-expressing metaphases were obtained from peripheral blood lymphocytes by standard methods. Briefly, cultures were grown for 72 hours in Eagle's minimal essential minimal medium, minus folic acid, supplemented with 5% fetal calf serum. Induction of FRA16D was with 0.5 uM aphidicolin (dissolved in 70% ethanol) added 24 hours before harvest (32). DNA clones were nick-translated with biotin- 14-dATP, pre-associated with 6 ug/ul total human DNA, hybridised at 20 ng/ul to metaphase preparations, and detected with one or two amplification steps using biotinylated anti-avidin and avidin-FITC as previously described (33). Hybridisation signal was visualised using an Olympus AX70 microscope fitted with single pass filters for DAPI (for chromosome identification), propidium iodide (as counterstain) and FITC. FRA16D-expressing chromosomes were scored for signal only when the width of the fragile site gap was greater than the width of one chromatid, so that signal was unambiguously proximal or distal to the gap (FIG. 3). Only fluorescent dots which touched chromatin were scored as signal—the few fluorescent dots which lay within the fragile site gap but did not touch proximal or distal segments were therefore not scored as signal since there was a possibility that they comprised non-specific background. Lambda clones which gave very poor FISH results (high non-specific hybridisation to other chromosomes) were not able to be scored with respect to the fragile site. This is likely to be due to the large amount of repetitive DNA within these particular clones—see below.

Tumour Cell Lines

The tumour cell lines LoVo, HT29, Kato III, SW480, AGS, MDA-MB-436 and LS180 were purchased from the American Type Culture Collection. LoVo and AGS cells were grown in Hams F12 medium with 2 mM L-glutamine, 10% fetal calf serum in 5% CO₂, Kato III cells were grown in RPMI1640 medium with 2 mM L-glutamine, 20% fetal calf serum in 5% CO₂, HT29 cells were grown in McCoy's 5a medium with 1.5 mM L-glutamine, 10% fetal calf serum in 5% CO₂, LS180 cells were grown in Eagle's minimal essential medium with 2 mM Lglutamine and Earle's salts and non-essential amino acids, 10% fetal calf serum in 5% CO₂, SW480 cells were grown in Leibovitz's L15 medium with 2 mM L-glutamine and 10% fetal calf serum, MDA-MB-436 cells were grown in Leibovitz's L 15 with 16 mg/ml glutathione and 0.026 units/ml insulin.

PCR Detection of Homozygous Deletion in Tumour Cell DNAs

PCRs for the detection of individual sequence tagged sites from across the FRA16D region were duplexed (34) with control PCRs from the dystrophin gene on the X chromosome (DMD Pm or DMD49, ref 35) or the APRT gene on chromosome 16 (33). This allowed verification that the PCR reaction was working in the absence of a FRA16D region PCR product (FIG. 4). Suitable PCR primers for Alu29, 17Sp6, Alu20, 178poly, 5. 1A6, RD69, IM7 were used or for 504CA, forward 5′- AACACAGCTCTTATCACATCC-3′ (SEQ ID No 6), reverse 5′-TGGCTGTAmGTCAGAACTG-3′ (SEQ ID No 7); while others were as given in database accessions, D16S518 (GenBank Z24645), Afma336yg9 (GDB 1222843), WI2755 (GenBank G03520), STSG-10102 (GenBank Z23147), D16S3029 (GDB 605884), WI-17074 (G22903), IM9 (GenBank R05832), D16S3096 (GenBank), D16S516 (GDB 200080). PCRs for GenBank AA368108 (forward 5′-TAATCCTCAGCCTCTAGAATGCCT-3′ (SEQ ID No 8), reverse 5′- GTATGATGATTTTCAGGGAGAAAC-3′) (SEQ ID No 9)and GenBank AA398024 (forward 5′-TGTCCTCAACTGATTCTTACAAAC-3 (SEQ ID No 10), reverse 5′-TCAATGGGTTAGGCACAGACC-3′ (SEQ ID No 11)) were derived from partial sequence analysis of BAC353B15. Control PCRs for FRA3B deletions were D3S1234 (GDB 186387), D3S1300 (GDB 188420) and D3S1841 (GDB 254090).

Results

Positional Cloning of FRA 16D

A contig of YAC clones was established in the 16q23.2 region between markers c306D2 and c307A12 which were found by FISH to map proximal and distal to FRA16D, respectively (FIG. 1B). The individual YACs from this contig were also used as hybridisation probes to further localise the fragile site. These experiments identified the YAC 801B6 as spanning FRA16D, and therefore this YAC was used as a source of DNA for subcloning the region to provide shorter DNA fragments for further refinement of the fragile site position. In addition, BAC clones were identified from the region to provide redundancy of cloned human DNA in an effort to avoid potential problems of instability of human DNA in YACs, as has previously been noted for other fragile site regions, including FRAXA (37), FRA1OB (38 and O. Handt, pers. comm.) and a Chinese hamster aphidicolin inducible fragile site region (39).

A pulsed-field gel restriction map of YAC 801B6 was constructed by using HincII restriction fragment subclones of the YAC for use as hybridisation probes (H13m, H22s, H23m, H29m and H40m) (FIG. 2A). The position of the BACs (379C2, 325M3 and 353B15) with respect to the YAC restriction map was determined by both the restriction mapping of the BACs and the positioning of common markers by PCR or hybridisation (FIG. 2A). The STS (D16S518, Afma336yg9, WI2755, STSG-10102 and D16S3029) content of the YACs and BACs was also determined to assist in map construction.

Subclone libraries of DNA from YAC 801B6 and BAC 325M3 were generated using the lambda vectors IGEM12 and IGEM11 (Promega), respectively and assembled into a contig by end-fragment hybridisation and restriction mapping. The integrity of the YAC restriction map was verified by comparison with that of the BACs, 325M3 and 353B 15. For the region between the BACs the integrity was verified by the use of long range PCR using human chromosomal DNA as template. (data not shown).

Localisation of FRA16D by Fluorescence In Situ Hybridisation (FISH)

There have been difficulties in determining the precise localisation of common chromosomal fragile sites using FISH (refs FRA3B (13, 40,41,42), FRA7G (18,19) and FRA7H (43). The FISH data have been interpreted as due to the fragile sites being spread out over long DNA sequences (eg 100's of kb) or that there are multiple fragile sites at a single locus. An alternative explanation is that the DNA in the immediate vicinity of the fragile site is not tightly ‘packaged’ into chromatin. We therefore chose to score only those chromosomes where the width of the gap or break at the FRA16D fragile site was greater than that of one chromatid (FIG. 3). This approach was intended to reduce the possibility that the ‘unpackaged fragile site DNA’ might be looping back over the distant side of the fragile site and therefore give a false ‘spanning’ signal—particularly for probes that are very close to or within the fragile site region. In addition, while the use of pre-reassociation in the hybridisation process dramatically improved the signal to noise ratio, it did render repeat rich regions poor hybridisation probes. This was particularly evident in the FRA16D region where there is an abundance of DNA repeat sequences of various kinds.

The results of the FISH experiments are plotted in FIG. 4. The closest clearly proximal probe to FRA16D is 11-44 while the closest unequivocally distal probe is 1433. These probes map at a distance of ˜200 kb apart. However, this 200 kb region includes consistent scatter of distal signal around 11-38 and 11-27 and the poor hybridisation between 1181 and 1511 (due to repetitive DNA content). Therefore this 200 kb defined by FISH analysis is likely to be the maximum sequence required to define FRA16D rather than provide any evidence that the fragile site is spread over such a distance.

Detection of Homozygous Deletion in Tumour Cell Lines

The FRA3B fragile site—FHIT gene intron 4 region is a frequent site of deletion in various types of cancer (8). Homozygous FRA3B deletions have been detected in various human adenocarcinoma cell lines including (gastric) AGS, Kato III; (breast) MDA- MB-436; (colon) LoVo, HT29, SW480 and LS180 (8). Since these deletions are somatic events that presumably occur as a result of exposure of these cells to certain environmental factors (11), we chose to analyse tumour cell lines which exhibit FRA3B deletions for the presence of homozygous deletion at the FRA16D locus.

STSs that were either mapped to the FRA16D region (FIG. 1) or generated from partial sequence analysis through the region (data not shown) were used to screen for homozygous deletion in various tumour cell line DNAs. The STSs were duplexed with a PCR from the dystrophin locus, as an internal control. The results for the analysis of one of the FRA16D region markers, STSG-10102 is shown in FIG. 4. Of the seven tumour cell lines tested, the stomach tumour cell line AGS was found to be homozygously deleted at STSG-10102 and a series of contiguous markers through the region, (Table 1) thus suggesting the presence of minimal deletions spanning the FRA16D region in each chromosome 16 present in the AGS cell line.

Detection of Heterozygous Deletion in AGS Tumour Cell Line DNA

The maximal extent of heterozygous deletion in the AGS tumour cell line in the FRA16D region was determined by genotyping polymorphic markers. The markers D16S518 and D16S3029 both gave two alleles indicating proximal and distal outer limits to the deletion of either chromosome 16 in AGS cells (FIG. 2A). The markers Afma336yg9 and 504CA were uninformative and therefore did not aid in delineating the limits of heterozygous deletion.

Open reading frames of 372 (FOR I), 423 (FOR II), 198 (FOR III) and 45 (FOR IV) amino acids were obtained for the respective mRNA sequences (FIG. 7). Identical N-termini, unique C-termini.

WW domains were identified by ProfileScan searches (at http://www.expasy.ch/prosite/).

Discussion

The region in which the chromosomal fragile site FRA16D is located has recently been shown to be associated with two types of chromosomal instability in cancer. In multiple myeloma, translocation of Ig loci into the 16q23 region causes the dysregulation of the c-MAF proto-oncogene on the affected allele. While these breakpoints are spread over at least 500 kb they bracket both the c-MAF gene and the FRA16D fragile site (1 and FIG. 1). The dysregulated expression results in elevated c-MAF mRNA levels, which is thought to contribute to neoplasia. These translocations were not identified by conventional cytogenetic analysis. Their detected frequency in multiple myeloma cell lines suggests an incidence of ˜25%.

Using representational difference analysis to identify differences between the genomes of normal and tumour cells, the FRA 16D region has also been shown to be the site of homozygous deletion in three different types (lung, ovary and colon) of adenocarcinoma (29). The commonly deleted region includes FRA16D, with the minimal deletion in colon tumour cell line corresponding almost exactly to the ˜200 kb region shown by our FISH studies to span the FRA16D fragile site. If common aphidicolin fragile sites confer susceptibility to mutagen induced DNA instability in cancer then tumour cell lines which have been shown to have such instability at one fragile site are likely to exhibit instability at another fragile site. By analysing tumour cell lines with known FRA3B deletions, we have found that the AGS cell line derived from a stomach cancer exhibits homozygous deletion spanning FRA16D. Heterozygosity of the flanking markers D16S518 and D16S3029 indicates that the chromosome 16 deletions are confined to the immediate vicinity of FRA16D.

Taken together these deletion data confirm the hypothesis that FRA16D is associated with specific chromosomal instability in cancer.

Given that the observed deletions are homozygous they are therefore likely to represent the loss of a negative function (eg tumour suppressor) rather than the gain of a tumour promoting function. If the analogy with the FRA3B locus holds then a gene either spanning or, at least partially, within the FRA16D commonly deleted region may contribute to neoplasia as a consequence of quantitative and/or qualitative effects of the deletion. Alternatively, the proximity of the FRA16D deletions to the c-MAF gene suggests that they have the potential to affect c-MAF expression. The FRA3B fragile site is associated with a region of ‘late’ replication (48) as are the ‘rare’ fragile sites FRAXA and FRAXE (49,50). Assuming that replication timing is affected by proximity to fragile site loci and, given the coupling of replication with transcription, the deletion of the FRA16D region may lead to an alteration in the timing, with respect to the cell cycle, of the expression of genes in the area—including c-MAF. ABBREVIATIONS BAC, bacterial artificial chromosome; DAPI, 4′, 6-diamindino-2-phenylindole; FISH, fluorescence in situ hybridisation; FITC, fluorescein isothiocyanate; LOH, loss of heterozygosity; FHIT, fragile histidine triad; FRA, fragile site locus; PCR, polymerase chain reaction; STS, sequenced tagged site; YAC, yeast artificial chromosome

### EXAMPLE 2

### DNA Sequencing of the FRA16D Fragile Site and the FOR Gene

Materials and Methods:

Cell Lines

Cell lines AGS, HCT116, HS578BST, HS578T, LS180, MDA-MB453 and T47D are from the Department of Cytogenetics and Molecular Genetics, WCH collection and were originally obtained from the American Type Culture Collection or the European Collection of Cell Cultures. AGS and LS180 cells were grown as described in Example 1. HS578BST cells were grown in OPTI-MEM with L-Glutamine, 0.0 mg/ml epidermal growth factor, 0.5 mg/ml hydrocortisone, 8% fetal calf serum in 5% CO₂. T47D, MDA-MB-453 and HS578T cells were grown in RPMI 1640 with L-glutamine, 10% fetal calf serum in 5% CO₂.

Large Scale Sequencing of FRA16D

Sequencing of the 270 kb region spanning FRA16D consisted of

a) Sonication libraries and

b) Nebulization libraries of BAC clones 325M3 and 353B 15 and

c) Restriction fragments of l clones (for sequencing between BAC 325M3 and BAC 353B 15).

a) Construction of Sonication Libraries:

For DNA sonication and cloning we modified the protocol from the Sanger Centre (http://www.sanger.ac.uk/Teams/Team53/sonication.shtml):

1 mg of each BAC-DNA were sonicated in 300 ml H₂O and 8 ml 10×Mung Bean Buffer (500 mM NaAc, 300 mM NaCl, 10 mM ZnSO₄ pH 5.0) on ice for 20 seconds using the Ultrasonic Inc. Heat Systems Sonicator W-225 (50% duty, 3.5 power). After reducing the volume to 80 ul, blunt ends were created with adding 40 U of Mung Bean Nucleases (Biolabs) and incubating the mixture at 30° C. for 25 minutes. The products were size fractioned on a 1% agarose gel and fragments ranging from 0.7-2 kb were extracted with the Qiaquick Gel Extraction Kit (Qiagen). 1500 ng of sonicated DNA (used in 500 ng aliquots) were ligated into pUC18-Sma vector (Pharmacia) at 16° C. overnight and transformed into Sure cells (electroporation-competent, Stratagene). 600 and 1500 clones of the sonication libraries of BAC 325M3 and 353B 15, respectively, were gridded on 96well plates and sequenced in one direction using the M13-forward primer. Sequences were assembled into contigs using the Staden Package (MRC) on an UNIX computer and edited in LASERGENE (Macintosh). For a selected number of clones additional sequences with the M13-reverse primer were retrieved and assembled. Additional sequencing primers were designed and PCR-products sequenced to close gaps between contigs.

b) Construction of Nebulization Libraries:

10 mg of each BAC DNA was mixed with 200 ml 10×TM buffer (500 mM Tris-HCl, pH 7.5, 150 mM MgCl₂), 1 ml sterile glycerol and H₂0 added to 2 ml. The mixture was pipetted into an IPI-nebulizer and nebulized at 10 psi for 45 seconds. The nebulized DNA was then precipitated, end-repaired, size-fractioned and cloned as described for the sonicated DNA. 300 and 500 nebulized clones of BAC 325M3 and 353B 15, respectively, were sequenced as described above and included in the assemblies. Subclones for sequencing of BAC 353B15 were picked randomly, whereas BAC 325M3 subclones were selected after hybridisation of specific l-clones of the tile path, made from the BAC 325M3.

c) Subcloning of restriction fragments of 1clones between 1-32 and 1-191 was done in pUC19-vector. Clones were sequenced with M13-forward and M13-reverse primers as well as with sequence-specific primers. In some cases subclones derived from specific restriction fragments were also subject to sonication, shotgun cloning and sequencing.

Sequencing was performed with the ABI Big Dye Terminator Kit from Perkin Elmer. In cases where sequencing with the Big Dye Terminator Kit failed, dRhodamine Terminator Kit was used, as recommended for GT-rich or homopolymeric regions by the ABI-DNA sequencing guide.

The final sequence was analysed using:

BLAST (http://www.ncbi.nlm.nih.gov/BLAST),

REPEATMASKER (http://ftp.genome.washington.edu/cgi-bin/RepeatMasker), and

GENSCAN (http://CCR-081.mit.edu/GENSCAN.html).

Northern Blot Hybridisation

Probes for hybridisation on multiple tissue northern blots from Clontech were:

a) exon 7 (186 bp), positions 690 through 876 of AF227526

b) part of exon 9A (779 bp), positions 1182 through 1961 of AF227527

c) exon 3-6A (366 bp), positions 291 through 657 of AF227528

d) part of exon 1A (163 bp), positions 298 through 461 of AF227529.

RNA Extraction

RNA was extracted from 1×10⁷ cells for each of the cell lines using the RNeasy Mini Kit from Qiagen: The cells were disrupted by addition of 600 ul lysis buffer RLT (supplied with the Kit). The lysed cells were homogenised by passing 5-10 times through a 21G (0.8×38 mm) needle attached to a 5 ml syringe. 600 ul of 70% ethanol were added and the samples were applied to RNeasy Mini Spin columns. Purification and elution of the samples were carried out according to the Kit's manual. 35-98 ug of total RNA were obtained.

RT-PCR

Reverse transcription was carried out in a 40 ul reaction volume using 12-33 ug of total RNA from cell lines AGS, HCT116, MDA.MB.453, LS180, T47D, HS578T and HS578BST, respectively, according to the product sheet of Gibco BRL Superscript RNAse H-Reverse Transcriptase Kit except for the addition of 20 U RNAse inhibitor (Rnasin, Promega) to the mixture.

Aliquots of 100 ng of cDNA were amplified in PCR reactions using various cDNA-primer combinations under standard PCR conditions (10 cycles of 94° C. for 30 sec, 60° C. for 30 sec, 72° C. for 30 sec, then 25 cycles of 94° C. for 30 sec, 55° C. for 30 sec, 72° C. for 30 sec).

Primers (5′-3′) used in RT-PCR were:

a) HHCMA-F (ATCTTGGCCTGCAGGAACATGGCA) (SEQ ID No 12)and wb85-F (TTATTCTGCA CTTTTCTGGCGGAG) (SEQ ID No 13), FORIII specific

b) FOR-ex3 (GAACAAGAAACTGATGAGAACGGA) (SEQ ID No 14)and wb85-F, FORIII specific

c) wb85-E12 (TTACTACGCCAATCACACCGAGGA) (SEQ ID No 15)and wb85-A (TGAATTAGCTCCAGTGACCACAAC) (SEQ ID No 16), common in FORI, FOR II and FOR III

5′ RACE

Complete 5′-ends of transcripts FORI, FORII and FORIII were determined by 5′ RACE experiments including first strand cDNA synthesis, purification, TdT tailing of the cDNA, PCR of dC-tailed cDNA and nested amplification according to the instruction manual of GibcoBRL. 1 ug of total RNA of cell lines HS578BST (normal) and T47D (tumour) were taken as templates. First strand cDNA synthesis was conducted with the following specific GSP 1primers:

| | | | |
|---|---|---|---|
| FORI | (coxido-R, | 5′-TTATTTCAGCACTCAGCTCAAAGTCAC-3′), | (SEQ ID No 17) |
| FORII | (HHCMA-B, | 5′-AGCAAAGAGACCTATGCCTAGCCCA-3′), | (SEQ ID No 18) |
| FORIII | (wb85-F, | 5′-TTATTCTGCACTTTTCTGGCGGAG-3′). | (SEQ ID No 13) |

PCRs of the dC-tailed cDNA were carried out with the GSP2-primers:

| | | | |
|---|---|---|---|
| FORI and FORII | (coxido-32, | 5′-ATATCTGTAAATCGATGGGACTCTG-3′), | (SEQ ID No 19) |
| FORIII | (wb85-A, | 5′-TGAATTAGCTCCAGTGACCACAAC-3′). | (SEQ ID No 16) |

Nested amplification was done with 5 ul of a 1:100 dilution of GSP2-PCR products and the GSP3-primers:

FORI and FORII (coxido-21,5′-ACATGAAGAGGCACATTCTTGGCCT-3′) (SEQ ID No 20)

and FORIII (wb85-E, 5′-TCCTCGGTGTGATTGGCGTAGTAA-3′) (SEQ ID No 21) in combination with the AUAP-primer (GibcoBRL) (SEQ ID No 21).

PCR-products were extracted with Qiaquick-Kit from agarose-gels after electrophoresis and sequenced directly with GSP3-primers and the primer tj96-C:

5′-GGAGGCAGCTCGTCCTCACTG-3′ (SEQ ID No 22).

3′ RACE

The 3′ RACE System for Rapid Amplification of cDNA Ends (Gibco BRL) was used to determine the alternatively spliced 3′-ends of transcripts encoding FORI. 3 mg of total RNA of the normal fibroblast cell line SF4635 and the tumour cell lines AGS and HCT116 were taken as templates for first strand synthesis. Instead of the adapter primer (AP) supplied with the kit, the following variant of this primer was used:

RACE-AP/VAR (5′-GGCCACGCGTCGACTAGTACGTACAGT {TTT }₅T-3′).

This allowed a nested PCR approach in the subsequent PCR reactions. The target cDNA was amplified with a primer overlapping the FORI exon 8/exon 9 boundary (5′-ACCAAGTCCATGGTTTCAGACTG-3′) and a RACE-NESTED primer (5′-CGTCGACTAGTACGTACAGT-3′). A second round of amplification was performed with exon 9 specific primer #9327 (5′-ACTGCCTGGTAGAAGGAGGTCACTTCT-3′) and the Abridged Universal Amplification Primer (AUAP, 5′GGCCACGCGTCGACTAGTAC-3′) supplied with the 3′-RACE kit. 1 ml of first round PCR product was used for the nested PCR reaction. Bands were cut out from agarose gels, purified with Gene Elute Gel Purification Kit (Sigma) and directly sequenced with primer #9327.

Chromosomal DNA sequences corresponding to the alternative exons 10, 10A and 10B were identified by BLAST searches of sequence databases. Exon 10 was located in GenBank AC009141, exon 10A in GenBank AF179633 and exon 10B in GenBank AF009145 (see FIGS. 6 and 10).

cDNA Sequence of FOR IV (AF227529)

The preliminary cDNA sequence of the FOR IV transcript is incomplete at its 5′ end at this stage. The sequence determined so far derives from overlapping EST-clones qf42f03× (AI149681) and tm79cll.xl (AI570665). The latter was sequenced additionally with the internal primer tj96-C (5′-GGAGGCAGCTCGTCCTCACTG-3′) (SEQ ID No 22).

Determination of Breakpoints in Cell Lines AGS and HCT116

Deletions in cell lines AGS and HCT116 were determined in duplex-STS-PCR reactions as described in example 1. All primers are listed from 5′→3′ in Table 1.

Four regions of homozygous deletion (referred to asHZD I-HZD IV) were detected in the AGS cell line. The proximal breakpoint for HZD I in AGS was narrowed down to 654 base pairs between STSs 16D-15/16D-36 (+) and 16D-1/16D-60 (−); the distal breakpoint of HZD I of 3962 base pairs is between STS 16D-70 (−) and 16D-47 (+). The proximal breakpoint for HZD II in AGS was narrowed down to 3030 base pairs between STSs 16D-57 (+) and 16D-67 (−); the distal breakpoint of HZD II of 1720 base pairs is between STS 16D-68 (−) and 16D-54 (+). The proximal breakpoint for HZD III in AGS was narrowed down to 209 base pairs between STSs 16D-51 (+) and 16D-55 (−); the distal breakpoint of HZD III of 5690 base pairs is between STS 16D-202 (−) and 16D-69 (+). The proximal breakpoint for HZD IV in AGS was narrowed down to 5179 base pairs between STSs 16D-30/16D-44 (+) and ETA1 (−); the distal breakpoint of HZD IV of ˜1500 base pairs is between STS IM7 (−) and 410S1 A (+).

Two regions of homozygous deletion (referred to asHZD I and HZD II) were detected in the HCT116 cell line. The proximal breakpoint for HZD I in HCT116 was narrowed down to 1835 base pairs between STSs 16D- 19 (+) and 16D-61 (−); the distal breakpoint of HZD I of 1549 base pairs is between STS 16D-62 (−) and qz19h11 (+). The proximal breakpoint for HZD II in HCT116 was narrowed down to 422 base pairs between STSs 16D-63 (+) and 16D-30 (−); the distal breakpoint of HZD II of 1513 base pairs is between STS 16D-66 (−) and 801A (+).

For determining the presence of exon 9 of FOR I (51 bp) in the AGS cell line a duplex PCR with genomic primers from the dystrophin gene (DMD) as described in example 1 was carried out with primers 8040/ 8041 (Table 1).

Results

DNA Sequence Spanning FRA16D

The DNA sequence spanning FRA 16D was determined by a combination of approaches. Firstly, a tile path of lambda subclones of YAC My801B6 and BAC 325M3 was restriction mapped with restriction endonucleases EcoRI, HindIII, BamHI and SacI in order to provide a reference framework with which to anchor the DNA sequence. Secondly, either whole BAC DNA preparations of BAC325M3 or BAC353B 15 or specific restriction fragments from the lambda subclone tile path were used as feedstock DNA for construction of random insert plasmid libraries. Sequences from the region between BAC325M3 and BAC353B15 (l subclone tile path 132 to 1191) were subjected to long range PCR and restriction digest analysis in order to verify the integrity of this sequence. Sequenced subclones were also ordered by hybridisation with individual lambda subclones from the minimal tile path. The DNA sequences were therefore assembled in a directed rather than random manner. This approach greatly assisted in the assembly of those regions that were rich in DNA repeats. The 270 kb contiguous sequence, with an average 4-fold sequence coverage, spanning FRA16D has been deposited in GenBank (accession number AF217490) (FIG. 6).

Relationship Between Deletion and Translocation Breakpoints and FRA16D

PCR analysis of sequence tags across the FRA16D region was used to refine the location of deletion breakpoints in the AGS and HCT116 tumour cell lines (FIG. 6). Both cell lines showed two distinct regions of homozygous deletion indicating a minimum of three deletion events on the two chromosome 16s in each cell line. Four regions of the FRA16D spanning sequence were particularly difficult to determine because of their composition (as evident by DNA polymerase pausing in sequencing). Each of these sequences coincided with breakpoint regions in HCT 116 or AGS tumour cell lines (FIG. 6). The unstable regions consisted of: 1) a polyA homopolymer region at 144 to 145 kb of DNA sequence AF217490; 2) an imperfect CT-repeat of 320 base pairs at position 177-178 kb; 3) an 8 kb region at position 191-199 kb encompassing a poly A homopolymer region followed by an AT-repeat; a polyT homopolymer repeat and two inverted (hairpin-forming) repeats and 4) a TG repeat followed by a homopolymer region (poly T) at 212-213 kb. This fourth sequence is located within a common breakpoint region for the AGS and HCT116 cell lines at 211.7 -219.9 kb of AF217490. PCR across each of the breakpoint regions in AGS and HCT116 cell lines using primers from positive flanking STSs failed to produce products suggesting that additional cryptic instability (e.g. inversions or amplifications) may also be present.

The locations of three previously identified multiple myeloma breakpoints (1) was determined by either scanning of partial database sequences (for ANBL 6 (5′, 3′) and JJN3) or by PCR of STSs on the tile path of lambda subclones spanning FRA16D (for MM.1).

Alternatively Spliced FOR Gene Spans Fragile Site FRA16D

Scanning of the 270 kb sequence spanning FRA16D by BLAST homology searches revealed a paucity of EST homologies. The exceptions were consecutive exons corresponding to sequences from the EST qg88f04.×1 (FIG. 6). These exons therefore locate FRA16D within a 260 kb intron. BLAST searches with the qg88f04.×1 EST sequence revealed considerable overlap with clusters of ESTs the longest available sequence of which was HHCMA56 (U13395). ESTs qg88f04 and HHCMA56 clearly have distinct 3′ end sequences and were therefore referred to as transcript I and transcript II. Another cluster of ESTs (transcript III) was found to share 5′ but not 3′ end sequences with transcripts I and II. A fourth cluster of ESTs (transcript IV) was found to share sequence homology, however this overlap is between the 5′ most sequences of transcripts I-III and the 3′ end of the EST cluster suggesting that it may represent an overlapping gene rather than another alternatively spliced transcript.

5′RACE experiments using mRNA from normal (HS578BST) and tumour (T47D) cells were utilised to extend and confirm the sequences of the clusters of GenBank EST sequences of transcripts I-IV and to determine the organisation of the alternatively spliced mRNAs which they represent. Transcripts I, II and III were found to have a common 5′ end indicating a common promoter. The exons shared and utilised in the alternatively spliced mRNAs were identified in BAC sequences AF217491, AF217492, AC009044, AC009280 and AC009129 (FIG. 6). The confinement of distribution of EST sequences amongst exons confirmed that the different transcripts were due to alternate splicing. Transcripts I-III share common initiation methionine with an adjacent 5′ Kozak translation initiation sequence and an upstream in-phase termination codon. The open reading frames code for proteins of 41.2kD, 46.7kD and 21.5kD respectively. Each of these open reading frames shares homology with the oxido-reductase family of proteins and therefore the gene has been named FOR (Fragile site FRA 16D Oxido-Reductase) with the alternative spliced transcripts I-III referred to as FORI, FORII and FORIII respectively.

Northern blot analysis with various FOR exon probes identified the 2.3 kb FORII transcript as the predominant and ubiquitously expressed mRNA with FORI and FORII mRNAs showing a similar pattern of expression. A DNA probe spanning the 5′ exons detected additional RNAs with a different tissue specific pattern. A cluster of ESTs (FIG. 6) with homology limited to exon 1 of the FOR gene was found from a BLAST search of the databases. This suggests that these transcripts (referred to as FORIV) might arise from a different promoter and may well constitute a different gene, the 3′ end of which overlaps with the 5′ end of FOR (FIG. 6). The 3′ end sequences of these ESTs contain a very short open reading frame (4.1kD) which is truncated with respect to that seen in the FOR transcripts. The complete FORI-FORIII mRNA and partial related transcript sequence (FOR IV) were determined from 5′RACE and RT-PCR products and deposited in GenBank (AF227526, AF227527, AF227528, AF227529).

FOR mRNA in Normal and Tumour Cells

RT-PCR and 5′-RACE were used to detect the various FOR transcripts in normal and tumour cells. Striking differences between the presence/absence of FOR I and FOR III transcripts was noted for the ‘normal’ fibroblast-like cell line HS578BST and various tumour cell lines (FIG. 4). 5′-RACE and RT-PCR products for transcript specific PCRs were sequenced to confirm the identity of the respective products. The sequence of the aberrant RT-PCR product from MDA-MB-453 cell line generated using a FORIII specific primer contains a retroviral element (HERV-H) 5′ of exons 5 and 6A of FOR (GenBank AF239665). In addition, one EST (qz23c04.×1) identified in database BLAST searches contains exons 1, 2 and 3 of FOR spliced at the 3′ end to another retroviral element LTR13. Homozygous deletion of FORI exon 9 detected in AGS tumour cells suggests that the gain of FORI transcript will not be a common event in tumour cells. Similarly, the loss of FORIII transcript is not common to all tumour cells as FORIII specific RT-PCR products were readily detected in both AGS and HCT116 cells (FIG. 15).

FOR Encoded Proteins

The alternative spliced mRNAs transcribed from the gene each show homology to the oxido-reductase superfamily of proteins. The open reading frames of the alternatively spliced FOR gene mRN As I-III have a common N-terminus which contains a WW domain (FIG. 10).

The WW domain is truncated in FORIV open reading frame, however since this mRNA appears to originate from a distinct promoter it may well be that an upstream reading frame is utilised in this mRNA. The open reading frame from the FOR III transcript retains the WW domain however it is truncated for approximately half the length of the oxido-reductase homology (FIG. 10).

Discussion

Identification of the FOR Gene Spanning FRA16D

Given the proposed role of the FHIT gene in mediating the biological consequences of FRA3B associated DNA instability in cancer cells we sought to identify the closest gene to 20 FRA16D which might mediate the biological effects of FRA16D associated DNA instability in cancer. Sequence analysis of the FRA16D spanning DNA sequence revealed the FOR gene as the sole transcript in the immediate vicinity of the minimal region of homozygous deletion in cancer cells. Alternative exons of this gene were found to flank both the FRA16D fragile site and the tumour cell deleted regions—the alternative exon 9 being deleted in the AGS cell line. No additional authentic transcripts from within the FOR gene intron were evident.

Differential Expression of Alternative Spliced and Aberrant FOR Transcripts in Normal and Tumour Cells

RT-PCR and 5′-RACE gave differing patterns of FOR transcript expression in various normal and tumour cell lines. It will be of interest to determine whether there are differences in the ratio of FOR transcripts which are consistent with the biological characteristics of various cell types e.g. neoplastic state or metastatic potential. It is unlikely that the presence of FOR I transcripts will be a common property of tumour cells since at least the AGS cell line is homozygously deleted for the FORI exon 9. Additional aberrant FOR transcripts, including sequences fused to retroviral LTRs, were detected in tumour cells.

It may well be that the ratio of the various FOR transcripts is perturbed by DNA instability in the region and that it is the resultant alteration in relative abundance of the various FOR encoded proteins which mediates the biological consequences of DNA instability at FRA16D. For example the homozygous deletion in AGS cells deletes exon 9 of the FOR I transcript and may have an effect on the stability of the FOR III transcript, however this deletion is unlikely to have any direct effect on the FORII transcript which terminates well outside the homozygously deleted region.

Possible Function of FOR and Role in Neoplasia

The FOR encoded proteins show sequence homology to the oxido-reductase family of proteins and contain a WW domain. Other members of this family of proteins include the YES proto-oncogene associated proteins and NEDD-ubiquitin ligases.

The open reading frame from the FORIII transcript retains the WW domain however it is truncated for approximately half the length of the oxido-reductase/ubiquitin-ligase homology (FIG. 10). The FORIII protein is therefore likely to be able to bind proteins that recognise the common FORI and FORII WW domain but not able to perform the enzymatic function encoded by the FORI and FORII proteins (possibly ubiquitination). Such characteristics make the FORIII protein a likely competitor of FORI and/or FOR II. Since ubiquitination facilitates the process of specific protein turnover FORIII could therefore act to prolong the half-life of its substrate by competing with FORI and/or FORII. Influencing this ratio may have therapeutic benefits. Thus the provision of reduced FORIII production by perhaps use of antisense to FORIII transcript may stabilise the balance. Alternatively over expression of FORI and/or FORII could tip the balance the other way.

WW domains are regions of protein-protein interaction that bind polyproline-rich motifs (PY domains) in specific partner proteins. Specificity in this interaction is determined by differences in particular amino acid in the various WW domains. Proteins known to bind to 30 WW domains include the YES proto-oncogene product and p53 binding protein-2 (Pirozzi et al., (1997) J. Biol. Chem 272, 14611-14616). Alteration in the relative levels of the FOR encoded proteins as a consequence of FRA16D associated instability is therefore likely to influence the biological function of the PY-motif containing-protein(s) which is (are) the normal binding partner that the FOR proteins share through their WW domain. The majority of deletions in the 16q23.2 region are heterozygous with the homozygous deletions being confined and limited in number. Cells which still have the capacity to produce FORII protein (from a normal chromosome 16 FOR allele) might have an elevated level of FORIII (through FRA16D associated deletion of the other chromosome 16 allele) and therefore have a selective “heterozygote” advantage.

The finding of aberrant FOR related transcripts spliced to retroviral RNA sequences in tumour cells that do not necessarily exhibit FRA16D homozygous deletion (e.g. MDA-MB-453, FIG. 15) suggests that dysfunction of the pathway involving the FOR WW domain could be a common event in neoplasia perhaps through other forms of FRA16D related DNA instability such as DNA insertion or translocation. Three out of five previously mapped multiple myeloma translocations (21) map within the FOR gene suggesting that DNA instability at the FRA16D locus and aberrant expression of the FOR gene may have a variety of roles to play in various forms of cancer.

For the purposes of working the invention a large number of references to pertinent methodologies are set forth in the following US patent documents:—U.S. Pat. No. 5,981,218 to Rio et al, U.S. Pat. No. 5,928,884 to Croce et al, U.S. Pat. No. 5,945,522 to Cohen et al, and U.S. Pat. No. 5,837,492 to Tavtigian et al. These documents are incorporated herein entirely specifically for purposes of permitting working of the invention.

For the purposes of this specification the word “comprising” means “including but not limited to”, and the word “comprises” has a corresponding meaning.

Reference in this specification to a document is not to be taken as an admission that the disclosure therein constitutes common general knowledge in Australia.

### References

1. Chesi et al. (1998) Blood 91, 44574463.

2. Yunis & Soreng (1984) Science 226, 1199-1204.

3. Hecht & Sutherland (1984) Cancer Genet.Cytogenet. 12, 179-181.

4. Simmers et al (1 987) Science 236, 92-94.

5. Simmers & Sutherland. (1998) Hum. Genet. 78, 144-147.

6. Sutherland (1988) Cancer Genet.Cytogenet. 31, 5-7.

7. Sutherland & Simmers. (1988) Hum. Genet. 78, 144-147.

8. Ohta et al. (1996) Cell 84, 587-597.

9. Friend et al (1986) Nature 323, 643-646.

10. Fearon et al. (1990) Science 247, 49-56.

11. Sozzi et al. (1996) Cell 85, 17-26.

12. Siprashvilli et al (1997) Proc. Natl. Acad. Sci. USA 94, 13771-13776.

13. Inoue et al. (1997) Proc. Natl. Acad. Sci. USA 94, 14584-14589.

14. Huebner et al. (1998) Ann. Rev. Genet. 32, 7-31.

15. Le Beau et al (1998) Genes Chromosomes Cancer 21, 281-289.

16. Sutherland et al. (1998) Trends Genetics 14,501-506.

17. Otterson et al. (1998) J. Natl. Cancer Inst. 18, 426-432.

18. Huang et al. (1998) Genes Chrom. Cancer 21, 152-159.

19. Huang et al (1998) Oncogene 16, 2311-2319.

20. Engelman et al. (1998). FEBS Lett 438, 403410.

21. Galbiati et al. (1998) The EMBO Journal 17, 6633-6648.

22. Coquelle et al. (1997) Cell 89, 215-225.

23. Papiris et al (1998) EMBO Jour 17, 325-333.

24. Chen et al. (1996). Cancer Research 56, 5605-5609.

25. Latil et al (1997) Cancer Research 57,1058 - 1062.

26. Yu et al. (1997) Cell 88, 367-374.

27. Maw et al (1992) Cancer Research 52, 3094-3098.

28. Horwitz et al. (1997) Am. J. Hum. Genet. 61, 871-881.

29. Watson et al (1999) Proceedings of the American Association of Cancer Research 40, 321 abs#2125

30. Callen et al (1992) Genomics 13, 1178-1185.

31. Gecz et al. (1997) Genomics 44, 201-213.

32. Sutherland et al. (1996) Fragile sites. In: R. A. Meyer (ed), Encyclopedia of Molecular Biology and Molecular Medicine, VCH, pp. 313-318, New York.

33. Callen et al. (1990) Ann. Genet. 33, 219-221.

34. Chamberlain et al. (1988) Nucleic Acids Res. 16, 1114-11156.

35. Beggs et al (1990). Human Genet. 86, 4548.

36. Richards et al. (1991) Genomics 10, 1047-1052.

37. Kremer et al (1991) Science 252, 1711-1714.

38. Hewett et al (1998) Molecular Cell 1, 773-781.

39. Palin et al. (1998) J. Cell Sci 111, 1623-1634.

40. Wilke et al (1996). Hum. Molec. Genet. 5, 187-195.

41. Boldog et al (1997) Hum. Molec. Genet. 6, 193-203.

42. Zimonjic et al (1997) Cancer Res 57, 1166-1170.

43. Mishmar et al (1998) Proc Natl Acad Sci USA 14, 8141-8146.

44. Pekarsky et al (1998)Cancer Res. 58, 3401-3408.

45. Glover et al (1998). Cancer Res. 58, 3109-3414.

46. Ji et al (1999) Cancer Res. 59, 333-339.

47. Sard et al (1999) Proc. Natl. Acad. Sci. USA 96, 8489-8492.

48. LeBeau et al (1998). Hum. Molec. Genet. 7, 755-761.

49. Hansen et al (1 997) Proc. Natl. Acad. Sci. USA, 94, 4587-4592.

50. Subramanian et al (1996) Am. J. Hum. Genet., 59, 407-416.

51. Jones et al (1995) Nature 376, 145-149.

Jones et al., (1994) Human Molecular Genetics 3:2123-2130.

Joneset al., (1995) Nature 376: 145-149, 1995.

Mimori et al., (1999) Proc. Natl. Acad. Sci. USA 96: 7456-7461.

Gnarra et al., (1994) Nature Genet. 7: 85-90.

Chesi et al., (1998) Blood 91, 4457-4463.

Pirozzi et al., (1997) J. Biol. Chem 272, 14611-14616

[image]

1. A method of detecting variation of a 16q23.2 target, said method comprising the steps of contacting target nucleic acid with one or more oligonucleotide suitable for use as hybridisation probe or nucleic acid amplification primer specific for binding the 16q23.2 specific target and ascertaining the binding of said oligonucleotide.

2. A method of detecting variation of a 16q23.2 target as in claim 1 wherein the 16q23.2 specific target is selected from one or more of the group comprising the FOR gene, the FRA16D site, or mRNA encoding FOR protein, and the 16q23.2 target reflects chromosomal rearrangements or mutations.

3. A method of detecting variation of a 16q23.2 target as in claim 2 wherein the 16q23.2 target is within the FOR gene and is selected from one or more of the group comprising exons 1A, 1, 2, 3, 4, 5, 6, 6A, 7, 8, 9, 9A, 10, 10A, 10B or introns located between two adjacent exons.

4. A method of detecting variation of a 16q23.2 target as in claim 3 wherein the 16q23.2 target within the FOR gene is selected as either the intron between exon 8 and 9, or the intron between exons 8 and 9A.

5. A method of detecting variation of a 16q23.2 target as in claim 2 wherein the 16q23.2 target is a pause site within the FRA16D.

6. A method of detecting variation of a 16q23.2 target as in claim 5 wherein the pause site is selected from the group consisting of i) a poly A homopolymer region at 144 to 145 kb of DNA sequence SEQ ID no 53, and ii) imperfect CT-repeat of about 320 base pairs at position 177-178 kb, iii) an approximately 8 kb region at position 191-199 kb encompassing a poly A homopolymer region followed by and AT repeat; a poly T homopolymer repeat and an two inverted repeats and iv) a TG repeat followed by a poly T homopolymer region at 212-213 kb.

7. A method of detecting variation of a 16q23.2 target as in claim 2 wherein the target is a breakpoint of one or more chromosomal rearrangements associated with a tumour.

8. A method of detecting variation of a 16q23.2 target as in claim 2 wherein the target is an oligonucleotide sequence including a point mutation or small DNA rearrangement associated with a tumour.

9. A method of detecting variation of a 16q23.2 target as in claim 2 wherein the 16q23.2 target is within the FOR gene and is selected from one or more of the group comprising exons 1A, 1, 2, 3, 4, 5, 6, 6A, 7, 8, 9, 9A, 10, 10A, 10B.

10. A method of detecting variation of a 16q23.2 target as in claim 2 wherein the target is any one of the splice variants FOR I, FOR II, FOR III or FOR IV.

11. A method of detecting variation of a 16q23.2 target as in claim 2 wherein the method consists of determining the level of expression of the FOR gene or any one or more exon thereof, by determining the level of mRNA expression using a probe specific for the FOR gene or exon thereof.

12. A method of detecting variation of a 16q23.2 target as in claim 11 wherein the target is selected from the group consisting of exons 6A, 1A, 9, 10, 10A, 10B and 9A and the method is used to give an indication of relative amounts of transcription of the FOR I, FOR IV, FOR II and FOR III splice variants.

13. A method of detecting variation of a 16q23.2 target as in claim 11 wherein the target is selected from the group consisting of the 6A, 9, 10, 10A, 10B and 9A exon and the method is used to give an indication of relative amounts of transcription respectively of the FOR I, FOR IV, FOR II and FOR III splice variants.

14. A method of detecting variation of a 16q23.2 target as in claim 13 wherein the method measures the level of mRNA expression of FORIII when compared to the level of FORII and/or FORI.

15. A method of detecting variation of a 16q23.2 target as in claim 2 using a plurality of distinctly binding oligonucleotides selected to bind to a plurality of corresponding chromosomally spaced apart targets to one or more change in said plurality of targets.

16. A method of detecting variation of a 16q23.2 target as in claim 15 wherein separate ones of the plurality of distinct oligonucleotides are held spatially separated on a physical support to provide allow for separately detecting the binding of each one of the distinctly binding oligonucleotides.

17. A method of detecting variation of a 16q23.2 target as in claim 2 including a preamplification step whereby the target nucleic acid is amplified before binding of the oligonucleotide.

18. A method of detecting variation of a 16q23.2 target as in claim 2 consisting of a PCR method wherein two oligonucleotides being PCR primers are used to contact the target followed by an amplifications step at least one of the oligonucleotides binding the target.

19. A method of detecting variation of a 16q23.2 target as in claim 2 wherein the physical form of the target nucleic acid is selected from the group consisting of chromosomal DNA, cDNA and mRNA.

20. A method of detecting variation of a 16q23.2 target as in claim 2 wherein the target is chromosomal and the method comprises detecting the heterozygosity or homozygosity for one or more variants in the 16q23.2 target.

21. A method of detecting variation of a 16q23.2 target as in claim 20 wherein the method includes the steps of providing a first set of one or more oligonucleotides and a second set of one or more oligonucleotides the first set of oligonucleotide being specific for a first variant of the target nucleic acid, the second set of oligonucleotides being specific for a second variant of the target nucleic acid, the first and second set of oligonucleotides being labelled so as to be capable of being distinguished, and the method comprising the steps of comparing the proportion of binding of the first and second set of oligonucleotides.

22. An isolated 16q23.2 nucleic acid molecule selected from the group consisting of
a) FRA16D site,
b) FOR gene,
c) mRNA of the FOR gene,
d) cDNA of the FOR gene,
e) variants of the above including, chromosomal rearrangements and mutations of sequences set out in a) to d) including those variants associated with cancers, and
f) nucleic acid sequences capable of hybridising specifically to any sequence of a to e or its complement under stringent hybridisation conditions.

23. An isolated 16q23.2 nucleic acid molecule as in claim 22 comprising an antisense molecule.

24. An isolated 16q23.2 nucleic acid molecule as in claim 22 capable of acting as a specific primers and probe for detecting cancer associated variations of DNA sequence selected from the group consisting of
g) a point mutation or small DNA rearrangement associated with a tumour.
h) a breakpoint of one or more chromosomal rearrangements associated with a tumour, and
i) a pause site within the FRA16D

25. A recombinant 16q23.2 nucleic acid molecule including a vector and a 16q23.2 nucleic acid sequence operably linked to a control element, wherein the 16q23.2 nucleic acid sequence is selected from the group consisting of:
a) FRA16D site,
b) FOR gene,
c) mRNA of the FOR gene,
d) cDNA of the FOR gene,
e) variants of the above including, chromosomal rearrangements and mutations of sequences set out in a) to d) including those variants associated with cancers, and
f) nucleic acid sequences capable of hybridising specifically to any sequence of a to e or its complement under stringent hybridisation conditions.

26. A recombinant 16q23.2 nucleic acid molecule as in claim 25 including one or more exons of the FOR gene, wherein the vector is an expression vector and the 16q23.2 nucleic acid sequence is aligned to produce or overproduce FOR proteins or variants thereof.

27. A recombinant 16q23.2 nucleic acid molecule as in claim 26 wherein the 16q23.2 nucleic acid sequence encodes a splice variant of the FOR protein selected from the group consisting of FOR I, FORII and FORIII.

28. A recombinant 16q23.2 nucleic acid molecule as in claim 27 wherein the 16q23.2 nucleic acid sequence encodes a splice variant of the FOR protein selected from the group consisting of FORII and FORIII.

29. A recombinant 16q23.2 nucleic acid molecule as in claim 25 wherein the recombinant vector produces an antisense molecule capable of blocking the expression of a splice variant of the FOR protein.

30. A recombinant 16q23.2 nucleic acid molecule as in claim 25 wherein the recombinant vector produces an antisense molecule capable of blocking the FORIII protein.

31. A purified protein encoded by a gene which is adjacent to or overlapping a chromosomal fragile site including a string of amino acids unique to a FOR protein as set out in SEQ ID No 32, SEQ ID No 33, SEQ ID No 34 or SEQ ID No 35, said amino acid string being at least 10 amino acids long and exhibiting at least 70% amino acid homology to any one of SEQ ID No 32, SEQ ID No 33, SEQ ID No 34 and SEQ ID No 35.

32. A purified protein encoded by a gene which is adjacent to or overlapping a chomosomal fragile site as in claim 31 wherein the amino acid string exhibits at least 90% homology to any one of SEQ ID No 32, SEQ ID No 33, SEQ ID No 34 and SEQ ID No 35.

33. A purified protein encoded by a gene which is adjacent to or overlapping a chomosomal fragile site as in either claim 31 or 32 wherein the amino acid string is at least 20 amino acids long.

34. A purified protein encoded by a gene which is adjacent to or overlapping a chomosomal fragile site as in either claim 31 wherein the protein has an oxidoreductase domain and/or one or more WW domains.

35. A purified protein encoded by a gene which is adjacent to or overlapping a chromosomal fragile site as in claim 34 having at least one WW domain having an amino acid string of 10 amino acids or greater with homology of greater than 70% with an amino sequence selected from the group comprising the region 16 to 49 or 57 to 90 of the FOR gene being the amino acid strings

| | |
|---|---|
| DELPPGWEERTTKDGWVYYANHTEEKTQWEHPKT | (SEQ ID No 4) |
| and | |
| GDLPYGWEQETDENGQVFFVDHINKRTTYLDPRL. | (SEQ ID No 5) |

36. A purified protein encoded by a gene which is adjacent to or overlapping a chromosomal fragile site as in claim 34 having an oxidoreductase domain having an amino acid string of 10 amino acids or greater with homology of greater than 70% with an amino sequence selected from the group comprising the region 130 to 156 or 204 to 247 or 293 to 324 of the FOR gene being the amino acid strings

| | |
|---|---|
| TGANSGIGFETAKSFALHGAHVILACR, | SEQ |
| | ID |
| LHVLVCNAATFALPWSLTKDGLETTFQVNHLGHFYLVQLLQDVL, | SEQ |
| | ID |
| YNRSKLCNILFSNELHRRLSPRGVTSNAVHPG | SEQ |
| | ID |

37. A purified FOR protein, or mutation, or splice variation thereof encoded by any two or more exons selected from the group comprising 1A, 1, 2, 3, 4, 5, 6, 6A, 7, 8, 9, 9A, 10, 10A, 10B joined.

38. A purified FOR protein as in claim 37 selected from the group consisting of FORI, FORII, FORIII, or FORIV.

39. A purified FOR protein as in claim 37 being FORI.

40. A purified FOR protein as in claim 37 being FORII.

41. A purified FOR protein as in claim 37 being FORIII.

42. An agent capable of selectively binding a FOR protein or fragment or variant thereof.

43. An agent capable of selectively binding a FOR protein as in claim 42, having a binding specificity to a splice variant of a FOR protein.

44. An agent capable of selectively binding a FOR protein as in claim 43 said agent capable of specifically binding to the C terminus of one of the splice variants selected from the group consisting of FOR I, FOR II, FOR III and FOR IV to distinguish between said one from others of the splice variants.

45. An agent capable of selectively binding a FOR protein as in claim 43 wherein the FOR protein is the FORIII splice variant and said agent also inhibits at least one intermolecular interaction with the FORIII.

46. An agent capable of selectively binding a FOR protein as in claim 42 wherein the agent is an antibody or fragment thereof.

47. A method of detecting variants of the FOR protein comprising contacting a test sample with one or more FOR protein binding agents capable of distinguishing between variants of the FOR protein, and detecting the binding of said agent.

48. A method of detecting variants of the FOR protein as in claim 47 the method including the quantitative measurement of one or more FOR protein variants in the test sample to give a measure of the relative amount of the one or more FOR protein variants in the test sample.

49. A method of detecting variants of the FOR protein as in claim 48 wherein the quantitative measurement is of FOR III and FORII and/or FORI to give a relative quantitative measurement of FOR III relative to FOR I or FOR II or both.

50. A recombinant host cell having stably inserted therein a DNA of any one of claims 25 to 30.

51. A recombinant host cell as in claim 45 capable of expressing a protein according to any or of claims 31 to 41.
